(19) United States
(12) Patent Application Publication
(10) Pub. No.: US 2026/0192006 A1
(43) Pub. Date: Jul. 9, 2026

(54) DISINFECTION AND VIRUS INACTIVATION DEVICE, AIR-CONDITIONING APPARATUS PROVIDED WITH THE SAME, AND DISINFECTION AND VIRUS INACTIVATION METHOD

(71) Applicant: Mitsubishi Electric Corporation, Tokyo (JP)

(72) Inventors: Akane NOMURA, Tokyo (JP); Akinori SHIMIZU, Tokyo (JP); Seiro YUGE, Tokyo (JP)

(73) Assignee: Mitsubishi Electric Corporation, Tokyo (JP)

(21) Appl. No.: 19/129,311

(22) PCT Filed: Nov. 22, 2022
(86) PCT No.: PCT/JP2022/043177

§ 371 Date: May 13, 2025

(51) Int. Cl. A61L 2/24; A61L 2/04; A61L 2/14; A61L 2/18; A61L 2/20; A61L 2/202; A61L 9/015; A61L 9/14; A61L 9/16; A61L 9/22; A61L 101/02; A61L 101/06; A61L 101/20; A61L 103/40; F24F 8/24

(52) U.S. Cl. CPC A61L 2/24; A61L 2/04; A61L 2/14; A61L 2/18; A61L 2/20; A61L 2/202; A61L 9/015; A61L 9/145; A61L 9/16; A61L 9/22; F24F 8/24; A61L 2101/02; A61L 2101/06; A61L 2101/20; A61L 2103/40; A61L 2202/11; A61L 2202/14; A61L 2202/15; A61L 2209/111; A61L 2209/134; A61L 2209/16

(57) ABSTRACT

A disinfection and virus inactivation device that performs a disinfection treatment or an inactivating treatment of microorganisms in a target space includes: a substance generation unit configured to generate a specific substance to perform the disinfection treatment or the inactivating treatment; a supply unit configured to generate an air flow and supply the specific substance generated from the substance generation unit into the target space; and an activity reducing unit configured to decrease an activity of the microorganisms. In the disinfection and virus inactivation device, the specific substance is supplied to the target space by the supply unit, while the activity of the microorganism is being decreased by the activity reducing unit.

### TECHNICAL FIELD

The present disclosure relates to a disinfection and virus inactivation device that performs disinfection and inactivates virus, an air-conditioning apparatus provided with the disinfection and virus inactivation device, and a disinfection and virus inactivation method.

### BACKGROUND ART

In the past, substances such as ions, ozone gas, hypochlorous acid water, and chlorine dioxide have been used to remove or inactivate bacteria, mold, and viruses. Ions or ozone gas is generated by discharge. Hypochlorous acid water or chlorine dioxide is generated, for example, through electrolysis or chemical formulation. By sending that substance into a room by using a fan, it is possible to perform disinfection as removal of bacteria that floats in the air in the room or to inactivate viruses therein.

Patent Literature 1 proposes a technique in which ions generated by discharge are supplied into a predetermined region in a room, to thereby disinfect the predetermined region. In Patent Literature 1, a human detecting sensor is installed to monitor the presence or absence of a person or persons in the room; and a normal disinfection operation is performed when a person or persons are present in the room, and a disinfection operation having a higher disinfection performance than the normal disinfection operation is performed when the room becomes unoccupied.

### CITATION LIST

### Patent Literature

Patent Literature 1: Japanese Unexamined Patent Application Publication No. 2016-114283

### SUMMARY OF INVENTION

### Technical Problem

In Patent Literature 1, bacteria or viruses in the room are removed by disinfection or inactivated using a specific substance such as ions or ozone gas. In order to prevent infectious diseases, disinfection or viral inactivation is required to be performed with a higher efficiency.

The present disclosure is made in view of the above circumstances, and relates to a disinfection and virus inactivation device that can highly efficiently disinfect an air-conditioning target space or inactivate viruses therein, an air-conditioning apparatus provided with the disinfection and virus inactivation device, and a disinfection and virus inactivation method.

### Solution to Problem

A disinfection and virus inactivation device according to an embodiment of the present disclosure is a disinfection and virus inactivation device that performs a disinfection treatment or an inactivating treatment of microorganisms in a target space and that includes: a substance generation unit configured to generate a specific substance to perform the disinfection treatment or the inactivating treatment; a supply unit configured to generate an air flow and supply the specific substance generated from the substance generation unit into the target space; and an activity reducing unit configured to decrease an activity of the microorganisms. In the disinfection and virus inactivation device, the specific substance is supplied to the target space by the supply unit, while the activity of the microorganism is being decreased by the activity reducing unit.

An air-conditioning apparatus according to another embodiment of the present disclosure includes: the above disinfection and virus inactivation device; and a heat exchanger configured to cause heat exchange to be performed between refrigerant that flows therein and air. The air-conditioning apparatus supplies the target space with an air flow that is regulated in temperature by the heat exchanger when passing through the heat exchanger and that contains the specific substance.

A disinfection and virus inactivation method according to still another embodiment of the present disclosure is a disinfection and virus inactivation method for performing a disinfection treatment or an inactivating treatment of microorganisms in a target space. The bacteria removing and virus inactivation method includes: generating a specific substance to perform the disinfection treatment or the inactivating treatment; and supplying the specific substance together with an air flow to the target space, while decreasing an activity of the microorganisms.

### Advantageous Effects of Invention

The disinfection or virus inactivation device, the air-conditioning apparatus, and the disinfection or virus inactivation method according to the embodiments of the present disclosure supply a specific substance into an air-conditioning target space, to thereby perform disinfection treatment or inactivation treatment of microorganisms, while reducing the activity of the microorganisms. Therefore, the disinfection or virus inactivation device, the air-conditioning apparatus, and the disinfection or virus inactivation method according to the embodiments of the present disclosure can efficiently perform disinfection to remove bacteria that is microorganisms, in the target space, or efficiently inactivate viruses in the target space.

### BRIEF DESCRIPTION OF DRAWINGS

FIG. 1 is an appearance diagram of the disinfection and virus inactivation device according to Embodiment 1.

FIG. 2 illustrates an example of a schematic section of the disinfection and virus inactivation device according to Embodiment 1.

FIG. 3 illustrates a utilization form of the disinfection and virus inactivation device according to Embodiment 1.

FIG. 4 is a block diagram of the disinfection and virus inactivation device according to Embodiment 1.

FIG. 5 illustrates a relationship between an ion concentration and a disinfection and virus inactivation effect.

FIG. 6 is a graph indicating an effect of the temperature on the activity of bacteria.

FIG. 7 is a graph indicating an effect of the temperature on the activity of viruses.

FIG. 8 is a graph indicating an effect of temperature in the case where bacteria are inactivated by ions.

FIG. 9 is a graph indicating an effect of the temperature in the case where viruses are inactivated by ions.

FIG. 10 is a control flowchart of the disinfection and virus inactivation device according to Embodiment 1.

FIG. 11 is an appearance diagram of a disinfection and virus inactivation device according to Embodiment 2.

FIG. 12 illustrates an example of a schematic section of the disinfection and virus inactivation device according to Embodiment 2.

FIG. 13 illustrates a utilization form of the disinfection and virus inactivation device according to Embodiment 2.

FIG. 14 is a perspective view of a grille body of the disinfection and virus inactivation device according to Embodiment 2.

FIG. 15 is a block diagram of the disinfection and virus inactivation device according to Embodiment 2.

FIG. 16 is an explanatory view for a locus detecting operation of the disinfection and virus inactivation device according to Embodiment 2.

FIG. 17 is an explanatory diagram for a disinfection and virus inactivation operation of the disinfection and virus inactivation device according to Embodiment 2.

FIG. 18 is a control flowchart of the disinfection and virus inactivation device according to Embodiment 2.

FIG. 19 is a schematic sectional view of an air-conditioning apparatus according to Embodiment 3.

FIG. 20 is a schematic view of the air-conditioning apparatus in FIG. 19 as seen from directly below.

FIG. 21 is an explanatory view for the disinfection and virus inactivation operation by the air-conditioning apparatus as illustrated by FIG. 19.

FIG. 22 is an operation flowchart of the air-conditioning apparatus according to Embodiment 3.

FIG. 23 illustrates a utilization form of the air-conditioning apparatus according to Embodiment 3.

### DESCRIPTION ACCORDING TO EMBODIMENTS

Embodiments of the present disclosure will be described with reference to the accompanying drawings in more detail. In each of figures in the drawings, components that are the same as or equivalent to those in a previous figure or previous figures are denoted by the same reference signs, and their descriptions will thus be omitted or simplified as appropriate. Furthermore, the shapes, sizes, and locations of components illustrated in each of the figures can be changed as appropriate within the scope of the present disclosure.

### Embodiment 1

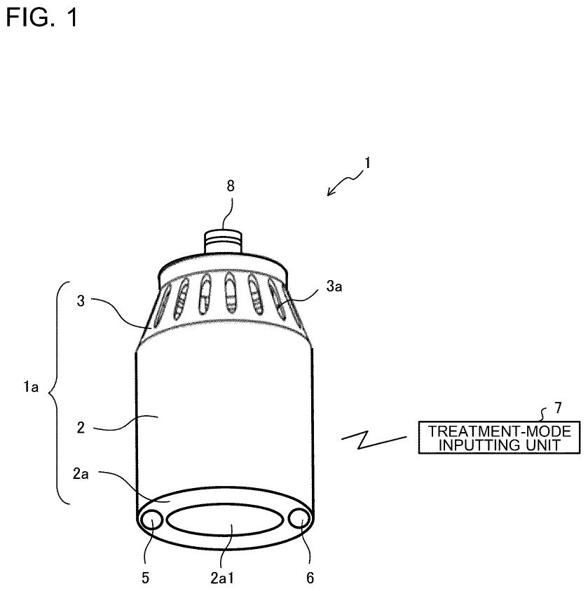

Regarding Embodiment 1, a disinfection and virus inactivation device 1 (see FIG. 1) for use in a space in an office or other rooms will be described by way of example.

Before description of the disinfection and virus inactivation device 1, routes of bacterial or viral infection will be described. It should be noted that targets to be subjected to disinfection or inactivation in the present disclosure are microorganisms including pathogenic microorganisms, such as bacteria or viruses. Infection routes are droplet infection, contact infection, airborne infection, etc.

Droplet infection refers to infection that occurs when mucous membranes of the mouth or the nose comes into contact with bacteria or viruses contained in “droplets” of saliva scattered by coughing, sneezing, or other actions. Wearing a mask is required to reduce droplet infection.

Contact infection refers to infection that occurs, through mucous membranes of the mouth or the nose, when an uninfected person touches surroundings that an infected person touches with his/her hand after having sneezed or coughed while putting his/her hand over his/her mouth.

Airborne infection refers to infection caused by particles of bacteria or viruses that are present in the air and smaller than droplets, specifically particles generated by coughing or sneezing or particles generated by evaporation of moisture of droplets. That is, airborne infection refers to infection caused by bacteria or viruses composed of smaller particles than droplets. Particles of bacteria or viruses that are smaller than droplets are, for example, particles that are generated as small particles during coughing or sneezing, or particles that generate when moisture in droplets evaporates after the droplets fly off into the air.

Regarding such infection as described above, it is possible to reduce droplet infection by wearing a mask. However, airborne infection and contact infection are infection that occurs after droplets are generated from a person, and it is hard for a person to take direct countermeasures against airborne infection and contact infection. Therefore, a technique for preventing airborne infection and contact infection is important.

The disinfection and virus inactivation device 1 according to Embodiment 1 is used mainly as a countermeasure against airborne infection. Microorganisms that fly off from an infected person are inhaled into an uninfected person who is present around the infected person, and as a result, the uninfected person becomes infected. In many cases, people become infected and develop a disease by inhaling the number of bacteria or viruses required to cause the disease, that is, the “minimum infective dose of bacteria or viruses”. Therefore, in order to reduce an infection rate, it is effective to reduce the number of microorganisms by treating flying-off microorganisms as early as possible, that is, to reduce the number of flying-off bacteria or viruses by removing the bacteria or inactivating the viruses as early as possible.

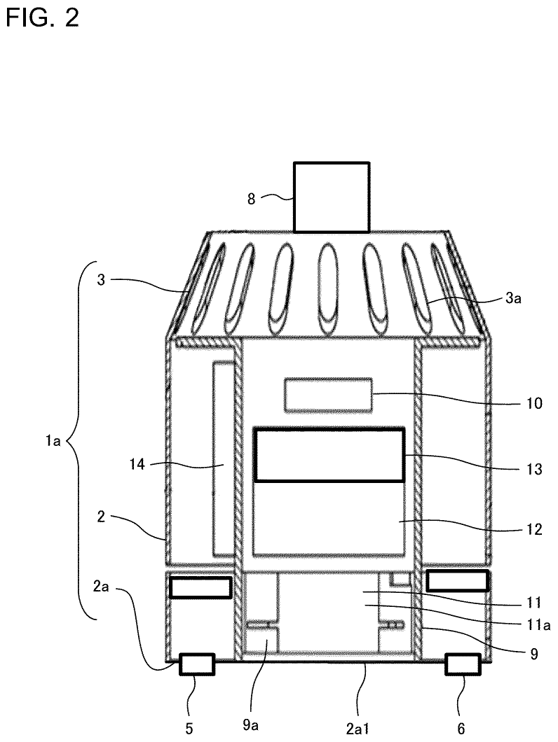

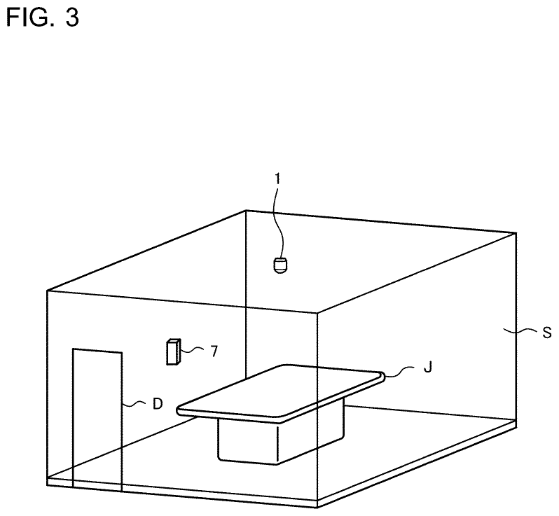

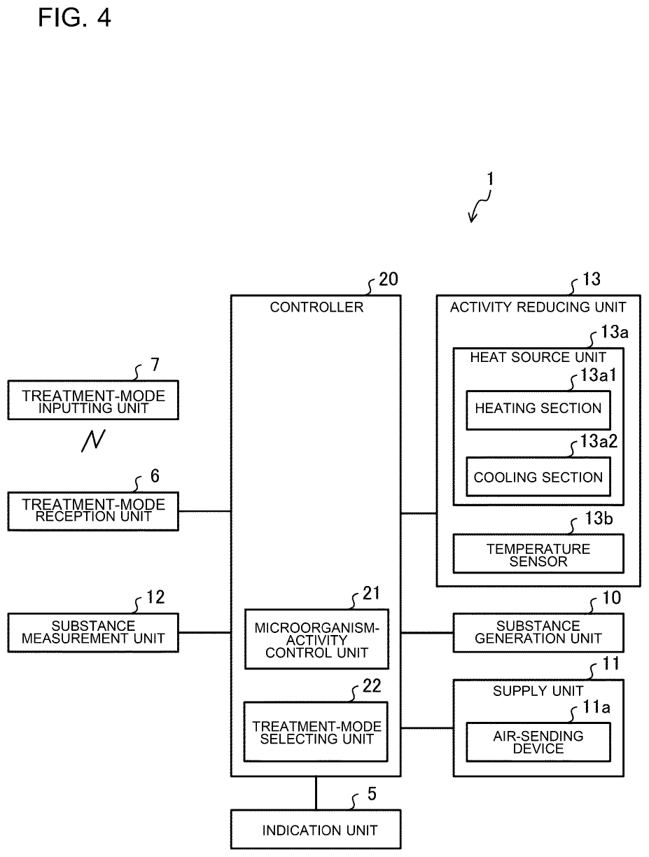

FIG. 1 is an appearance diagram of the disinfection and virus inactivation device 1 according to Embodiment 1. FIG. 2 illustrates an example of a schematic section of the disinfection and virus inactivation device 1 according to Embodiment 1. FIG. 3 illustrates a utilization form of the disinfection and virus inactivation device 1 according to Embodiment 1. In the following, directions such as up and down are based on the state of installation of the disinfection and virus inactivation device 1 as illustrated in FIGS. 1 to 3. FIG. 4 is a block diagram of the disinfection and virus inactivation device 1 according to Embodiment 1.

The disinfection and virus inactivation device 1 is a device that is installed at a high position such a ceiling in a target space S to be subjected to bacterial removal or viral inactivation, and that supplies the target space S with a specific substance for use in treatment for microorganisms in the target space S, that is, microorganisms that floats the in the target space S. It should be noted that the microorganisms mean bacteria or viruses. In the case where the microorganisms are bacteria, the treatment for the microorganisms is treatment by which the bacteria are removed, and in the case where the microorganisms are viruses, the treatment for the microorganisms is treatment by which the viruses are inactivated.

The target space S is a closed space that a person or persons enter and exit, for example, a space, such as an office, partitioned by a partition and provided with a door D through which a person or persons enter and exit the space. In the target space S, equipment J is installed. The equipment J refers to fixtures and fittings that are present in the target space S. The equipment J refers to a table and a counter in an indoor space in an ordinary house or other buildings, and the equipment J in an indoor space in an office or other buildings refers to daily-use tools, such as desks and racks, which are present in the space.

As illustrated in FIGS. 1 and 2, the disinfection and virus inactivation device 1 has a housing 1a that includes a cylindrical tubular portion 2, an annular lower surface portion 2a attached to a lower part of the tubular portion 2, and an annular upper surface portion 3 covering an upper-end opening of the tubular portion 2. A ferrule 8 that is connected to a fixture attached at a high location such as a ceiling is attached to an upper end portion of the housing 1a. The disinfection and virus inactivation device 1 is configured such that commercial power is supplied to a power supply device (which will be described later) via the ferrule 8 when the ferrule 8 is connected to the fixture.

The upper surface portion 3 of the housing 1a has a plurality of air inlets 3a that are spaced from each other in a circumferential direction, and through which air is taken in from the outside. A filter (not illustrated) is detachably provided on an inner surface side of each of the air inlets 3a. An air-passage forming component 9 communicating with the air inlets 3a is fixed in the tubular portion 2. The air-passage forming component 9 has an air passage 9a therein. An upstream side of an air passage 9a communicates with the air inlets 3a. A downstream side of the air passage 9a communicates with an exhaust outlet 2a1 formed by an opening in the center of the lower surface portion 2a. The outer shape of the housing 1a is not limited to the aforementioned shape but may be any outer shape, and, for example, the housing 1a is shaped such that the tubular portion 2 is formed in the shape of a tube that is rectangular in section.

An indication unit 5 and a treatment-mode reception unit 6 (which will be described later) are attached to the lower surface portion 2a of the housing 1a. The indication unit 5 is configured to indicate an operation state of the disinfection and virus inactivation device 1. The indication unit 5 and the treatment-mode reception unit 6 may be provided on the same mounting board or may be provided on respective mounting boards. In the case where the indication unit 5 and the treatment-mode reception unit 6 may be provided on the same mounting board, manufacturing costs can be reduced. Referring to FIG. 1, the disinfection and virus inactivation device 1 further includes a treatment-mode inputting unit 7 that is connected to the treatment-mode reception unit 6 in the housing 1a such that the treatment-mode inputting unit 7 can communicate with the treatment-mode reception unit 6.

In the housing 1a, a substance generation unit 10, a supply unit 11, a substance measurement unit 12, an activity reducing unit 13, and a main substrate 14 are provided.

Components of the disinfection and virus inactivation device 1 will be described.

### Description of Substance Generation Unit 10

The substance generation unit 10 generates a specific substance to perform a disinfection treatment or a virus inactivating treatment. The specific substance is ions, ozone gas, chlorine dioxide, hypocholorous acid water, or other substances that can destroy or inactivate microorganisms including pathogenic microorganisms that a person retains. The substance generation unit 10 is attached to an inner wall of the air-passage forming component 9. In Embodiment 1, the substance generation unit 10 includes a discharge mechanism configured to generate ions. The discharge mechanism is provided to face the air passage 9a in the housing 1a. The discharge mechanism is configured as a unit including a discharge unit, an electrode cover covering the discharge unit, and a casing in which the discharge unit and the electrode cover are provided. Furthermore, the discharge mechanism includes a control circuit board on which a high-voltage generation circuit or other circuits are mounted. The control circuit board is provided with a connector through which electric power is supplied from the outside.

The discharge unit includes discharge electrodes and ground electrodes. The discharge electrodes are each a wire electrode, and the ground electrodes are each a plate electrode. To be more specific, the discharge unit is configured such that a plurality of wire electrodes and a plurality of plate electrodes are alternately arranged. The discharge unit is given a high voltage from the high-voltage generation circuit. The high-voltage generation circuit includes a power reception unit that receives electric power from a commercial power source, and the high-voltage generation circuit converts, into a high voltage, electric power received by the power reception unit through a connector or an electric wire and gives the high voltage to the discharge unit. The discharge unit causes discharge by applying a high voltage given from the high-voltage generation circuit between the discharge electrode and the ground electrode, and causes ions to be generated in the air. Although it is described above regarding this example that the discharge electrodes are each a wire electrode and the ground electrodes are each a plate electrode, this is merely an example. However, both the discharge electrodes and the ground electrodes may be wire electrodes, needle electrodes, plate electrodes, or brush electrodes.

### Description of Supply Unit 11

The supply unit 11 includes an air-sending device 11a that generates an air flow. The supply unit 11 causes the air-sending device 11a to generate an air flow, and supplies, into the target space S, the specific substance generated by the substance generation unit 10. The air-sending device 11a is provided downstream of the substance generation unit 10 in the air passage 9a. Thus, the supply unit 11 mixes, within a fan of the air-sending device 11a, the specific substance generated by the substance generation unit 10 with air, and blows the mixture of the specific substance and the air to the outside of the housing 1a, with the ion concentration in the air uniformed.

The air-sending device 11a includes an air-sending fan and a motor that drives drive the fan. The fan is provided on an exit side of the air passage 9a. The fan is supported on an inner wall of the tubular portion 2 of the housing 1a such that the fan is located on a central axis of the air passage 9a. As the fan, an axial-flow propeller fan is employed to generate a large air volume of air flow. Furthermore, as the motor for the fan, an AC capacitor motor is employed. When the fan is driven in the air-sending device 11a, air around the tubular portion 2 is radially sucked into the tubular portion 2 through the air inlets 3a and flows into an inlet of the air passage 9a. The flow of the air flow that has flowed into the inlet of the air passage 9a changes from the flow of air flow in a radial direction to the flow of an air flow in an axial direction. Then, the air flow that flows through the air passage 9a in the axial direction is blown out to the outside of the housing 1a through the exhaust outlet 2a1.

The substance generation unit 10 is provided upstream of the supply unit 11. Because of provision of this configuration, the disinfection and virus inactivation device 1 can mix, within the fan of the air-sending device 11a, the specific substance generated by the substance generation unit 10 with air and supplies the mixture of the specific substance and air into the target space S that is located outside the housing 1a, with the ion concentration in the air uniformized. The substance generation unit 10 may be provided downstream of the supply unit 11. Many of the specific substances generated by the substance generation unit 10 are oxidizing substances such as ozone gas, and cause corrosion and deterioration of the fan of the air-sending device 11a and the motor configured to drive the fan. However, by virtue of the above configuration, the disinfection and virus inactivation device 1 can prevent corrosion and deterioration of the air-sending device 11a, because the specific substances generated by the substance generation unit 10 do not pass through the air-sending device 11a.

### Description of Substance Measurement Unit 12

The substance measurement unit 12 includes an ion sensor that measures a discharge product in the air. The ion sensor is provided downstream of the substance generation unit 10 in the flow direction of air in the air passage 9a. As the ion sensor, a coaxial bicylindrical sensor that measures positive ions or negative ions in the air is employed. The ion sensor can thus measure positive ions and negative ions simultaneously and can carry out high-precision measurement in a wide concentration range of 100,000 to 3,000,000 (ions/cm³). The result of the measurement by the substance measurement unit 12 is output to a controller 20 (which will be described later). In the case where the specific substance generated by the substance generation unit 10 is ozone, the substance measurement unit 12 is an ozone gas sensor that measures ozone in the air.

### Description of Activity Reducing Unit 13

The activity reducing unit 13 is a unit configured to reduce the activity of microorganisms. The activity reducing unit 13 is provided between the substance generation unit 10 and the substance measurement unit 12 in the flow direction of air in the air passage 9a. The location of the activity reducing unit 13 is not limited to the above location, and the activity reducing unit 13 may be provided at any location as long as the activity reducing unit 13 is provided at a location where the activity reducing unit 13 can reduce the activity of the microorganisms. The activity reducing unit 13 reduces the activity of the microorganisms by controlling the temperature of the microorganisms. To be more specific, the activity reducing unit 13 reduces the activity of the microorganisms by controlling the temperature of the microorganisms such that the temperature thereof is set to a temperature at which the activity of the microorganisms decreases. The disinfection and virus inactivation device 1 brings about improvement in an effect of removing bacteria or inactivating viruses by reducing the activity of the microorganisms with the activity reducing unit 13 (such an effect being hereinafter referred to as a “disinfection and virus inactivating effect”). A mechanism of improvement in the disinfection and virus inactivating effect by a decrease in the activity of the microorganisms will be described in detail later.

It is possible to perform the control of the temperature of the microorganisms by controlling the temperature of an air flow that is supplied from the housing 1a to the target space S, that is, the temperature of an air flow that comes into contact with the microorganisms. The control of the temperature of the microorganisms may also be performed by, instead of control of the temperature of the air flow, control of the temperature of a place in which the microorganisms are present, for example a surface of the equipment J at which the specific substance comes into contact with the microorganisms. Furthermore, the control of the temperature of the microorganisms may also be performed by control of the temperature of a place where the specific substance is generated.

Instead of controlling the temperature of the microorganisms, the activity reducing unit 13 may reduce the activity of the microorganisms, for example, by spraying an appropriate chemical for the microorganisms toward the target space S. Although the activity reducing unit 13 can thus adopt various methods in order to reduce the activity of the microorganisms, the following description is made by referring to by way of example the case where the activity reducing unit 13 reduces the activity of the microorganisms by controlling the temperature of the air flow.

As illustrated in FIG. 4, the activity reducing unit 13 includes a heat source unit 13a and a temperature sensor 13b. As illustrated in FIG. 2, the heat source unit 13a is provided in the air passage 9a. The heat source unit 13a is provided in the airflow path of the air flow generated by the air-sending device 11a. The heat source unit 13a includes a heating section 13a1 and a cooling section 13a2 and controls the temperature of the air flow with heating energy of the heating section 13a1 or cooling energy of the cooling section 13a2. To the heating section 13a1, a resistance heating method is applied in which heat is generated, for example, by causing an electric current to flow through a heating element made of metal or non-metal (e.g. resin). To the cooling section 13a2, a Peltier cooling method is applied. The heating section 13a1 and the cooling section 13a2, which adopt the above methods, can be made compact and can promptly perform a temperature control. The heat source unit 13a can control the temperature of the air flow in the range of approximately 10 to 35 degrees C. at about ambient temperature or, at lowest, in the range of 20 to 30 degrees C. The temperature sensor 13b includes a thermocouple or other components and measures the temperature of an air flow that passes through the inside of the housing 1a. The temperature sensor 13b measures the temperature of the air flow that is supplied from the housing 1a to the target space S.

### Description of Main Substrate 14

On the main substrate 14, a controller 20, a power supply device and other devices are mounted. The controller 20 controls the entire disinfection and virus inactivation device 1, and the power supply device supplies power to each of components. The main substrate 14 is fixed to a side wall of the air-passage forming component 9 of the tubular portion 2 of the housing 1a. The controller 20 is, for example, a microprocessor unit, and includes a CPU, a RAM, a ROM, etc., and the ROM stores a control program or other programs. The CPU and the control program form a microorganism-activity control unit 21 and a treatment-mode selecting unit 22 (which will be described later).

The controller 20 performs a disinfection and virus inactivation operation in which the activity of the microorganisms is reduced, and the microorganisms in the target space S are treated by supplying the specific substances generated by the substance generation unit 10 to the target space S. Thus, the disinfection and virus inactivation device 1 improves the disinfection and virus inactivating effect by supplying the specific substance to the target space S, with the activity of the microorganisms reduced.

To the controller 20, as illustrated in FIG. 4, the treatment-mode reception unit 6, the substance generation unit 10, the supply unit 11, the substance measurement unit 12, the activity reducing unit 13, and the indication unit 5 are electrically connected by lead wires. The controller 20 includes a microorganism-activity control unit 21 and a treatment-mode selecting unit 22. The microorganism-activity control unit 21 controls the heat source unit 13a of the activity reducing unit 13 such that the temperature of the microorganisms reaches a temperature at which the activity of the microorganisms is reduced. While reducing the activity of the microorganisms by controlling the activity reducing unit 13, the controller 20 supplies the specific substance to the target space by controlling the supply unit 11.

The microorganism-activity control unit 21 has a disinfection mode and a virus inactivating mode as treatment modes, and controls the heat source unit 13a of the activity reducing unit 13 to cause the temperature measured by the temperature sensor 13b to reach a temperature set in advance according to a treatment mode. The temperature at which the activity of the microorganisms decreases and the modes will be described later.

The treatment-mode selecting unit 22 is a unit configured to select a treatment mode of the microorganism-activity control unit 21. The treatment-mode selecting unit 22 selects a treatment mode in response to a user's input operation. Specifically, the treatment-mode selecting unit 22 obtains, via the treatment-mode inputting unit 7 and the treatment-mode reception unit 6 (which will be described later), a treatment mode input from the user and selects the obtained treatment mode. The controller 20 performs the disinfection and virus inactivation operation with the microorganism-activity control unit 21 in the treatment mode selected by the treatment-mode selecting unit 22.

Furthermore, the controller 20 controls the indication unit 5 based on the result of the measurement by the substance measurement unit 12. Specifically, when detecting, based on the result of the measurement by the substance measurement unit 12, that the concentration of the specific substance is lower than or equal to a set concentration set in advance, the controller 20 causes the substance generation unit 10 to stop the operation thereof and causes the indication unit 5 to light. The controller 20 controls the indication unit 5 to cause the indication unit 5 to be in a lighting state indicating an abnormality in the substance generation unit 10. Thus, the disinfection and virus inactivation device 1 can make a notification indicating occurrence of the abnormality.

Furthermore, the controller 20 controls the indication unit 5 based on the result of the measurement by the temperature sensor 13b of the activity reducing unit 13. Specifically, when detecting, based on the result of the measurement by the temperature sensor 13b, that the temperature of the air flow is not the temperature set in advance, the controller 20 causes the heat source unit 13a of the activity reducing unit 13 to stop the operation thereof and causes the indication unit 5 to light. The controller 20 controls the indication unit 5 to cause the indication unit 5 to be in a lighting state indicating an abnormality in the activity reducing unit 13. Thus, the disinfection and virus inactivation device 1 can make a notification indicating occurrence of the abnormality.

### Description of Treatment-Mode Inputting Unit 7

The treatment-mode inputting unit 7 is a unit that enables the user to input the disinfection mode or the virus inactivating mode to the unit as the treatment mode of the microorganism-activity control unit 21. The treatment-mode inputting unit 7 is provided separate from the housing 1a. The treatment-mode inputting unit 7 is, for example, an infrared remote control, and includes an operating unit on which a person performs an input operation and a communication unit configured to communicate with the treatment-mode reception unit 6 (which will be describe later). The operating unit is a switch, buttons, or other components. The communication unit is a communication interface that conforms to various standards and uses a pulse-position modulation (PPM) signal or other signals for communication. The treatment-mode inputting unit 7 may be a device such as a smartphone or a tablet in which an application for inputting a treatment mode is installed. When a treatment mode is input to the treatment-mode inputting unit 7 from the operating unit by the user, the treatment-mode inputting unit 7 sends the input treatment mode to the treatment-mode reception unit 6 via the communication unit.

### Description of Treatment-Mode Reception Unit 6

The treatment-mode reception unit 6 is a unit configured to receive a treatment mode from the treatment-mode inputting unit 7 and send the treatment mode to the treatment-mode selecting unit 22. The treatment-mode reception unit 6 is, for example, an infrared reception unit. By generating a voltage upon reception of an infrared signal from the treatment-mode inputting unit 7, the treatment-mode reception unit 6 receives the sent treatment mode and sends the treatment mode to the treatment-mode selecting unit 22.

It should be noted that the disinfection and virus inactivation operation is started when the treatment mode is input from the treatment-mode inputting unit 7. Specifically, the control is performed as follows. When the treatment mode is input from the treatment-mode inputting unit 7, an operation start command including the treatment mode is sent from the treatment-mode inputting unit 7 to the treatment-mode reception unit 6. When the treatment-mode reception unit 6 receives the operation start command including the treatment mode, the disinfection and virus inactivation device 1 starts the disinfection and virus inactivation operation in the received treatment mode. It should be noted that the start of the disinfection and virus inactivation operation is not necessarily triggered by input of the treatment mode from the treatment-mode inputting unit 7, but the disinfection and virus inactivation operation may be started by pressing an operation start button that is provided at the treatment-mode inputting unit 7. In this case, the treatment-mode selecting unit 22 may select, for example, a treatment mode set as a default setting or may re-select a treatment mode selected previously.

### Description of Indication Unit 5

The indication unit 5 is attached to an outer wall surface of the lower surface portion 2a of the housing 1a as an electronic component for providing information. The indication unit 5 is, for example, a light-emitting diode (LED) that gives various types of information. The indication unit 5 indicates an operation state of the disinfection and virus inactivation device 1, through a lighting state of the light-emitting diode. The indication unit 5 can change the lighting state through an appropriate combination of the color of light emitted by the light-emitting diode and a lighting pattern such as blinking or lighting. By changing the lighting state of the light-emitting diode, the indication unit 5 can indicate whether the current treatment mode is the disinfection mode or the virus inactivating mode, or make a notification indicating an abnormality.

### Mechanism of Improvement in Disinfection and Virus Inactivating Effect by Specific Substance

In general, the specific substance exhibits the disinfection and virus inactivation effect, when its concentration reaches or exceeds a certain threshold. The specific substance is rapidly improved in disinfection effect when the concentration further increases.

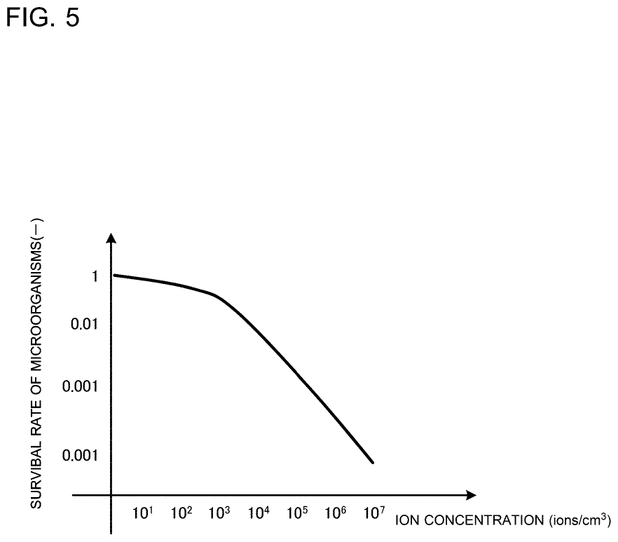

FIG. 5 illustrates a relationship between an ion concentration and the disinfection and virus inactivation effect. In FIG. 5, the horizontal axis represents an ion concentration (ions/cm³), and the vertical axis represents the survival rate (−) of the microorganisms. As indicated in FIG. 5, the survival rate of the microorganisms begins rapidly falling at an ion concentration of 10³ (ions/cm³), indicating the onset of the disinfection and virus inactivation effect. Moreover, the disinfection and virus inactivation effect is further improved as the ion concentration becomes higher than 10³ (ions/cm³). For this reason, the substance generation unit 10 is configured to generate ions in ion concentrations higher than or equal to 10³ (ions/cm³).

### Description of Mechanism of Improvement in Disinfection and Virus Inactivation Effect by Reduction of Activity of Microorganisms

The disinfection and virus inactivation device 1 is improved in the disinfection and virus inactivation effect by control of the temperature of the air flow that is performed by the activity reducing unit 13. The following description is made with respect to the mechanism of improvement in the disinfection and virus inactivation effect by temperature control.

When the activity of microorganisms such as bacteria or viruses decreases, the proliferation potency of the bacteria, infectivity of the viruses, and restorative ability of bacterial or viral structures decrease, the microorganisms are easily affected by disturbing factors. That is, when the activity of the microorganisms decreases, the microorganism are more prone to disinfection or viral inactivation. Furthermore, when the microorganisms come into contact with the specific substance such as ions, ozone gas, chlorine dioxide, or hypocholorous acid water, they are more prone to disinfection or viral inactivation. Therefore, in order to improve the disinfection and virus inactivation effect, it is effective to reduce the activity of the microorganisms and bring the specific substance into contact with the microorganisms the activity of which is reduced.

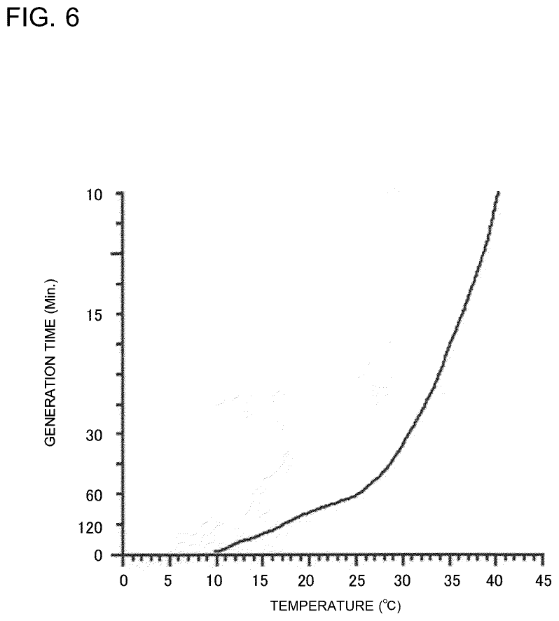

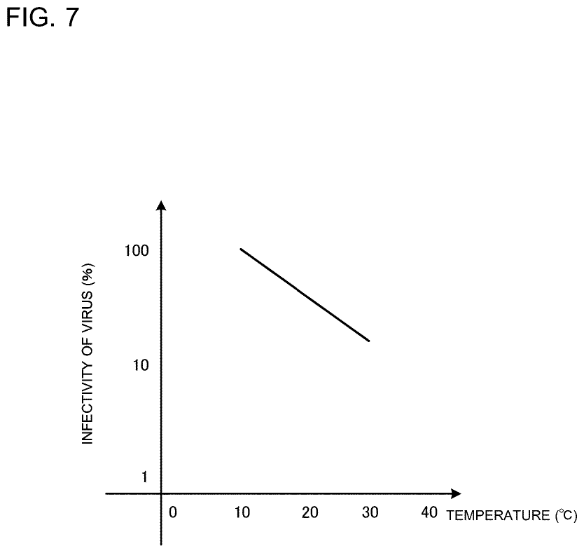

FIG. 6 is a graph indicating an effect of the temperature on the activity of bacteria. In FIG. 6, the horizontal axis represents the temperature (degree C.) of the microorganisms, and the vertical axis represents generation time (min) in the bacteria. The generation time (min) is the time required for the bacteria to divide. The graph of FIG. 6 is a graph of the case where the bacteria are Escherichia coli bacteria. FIG. 7 is a graph indicating an effect of the temperature on the activity of viruses. In FIG. 7, the horizontal axis represents the temperature (degree C.) of the microorganisms, and the vertical axis represents the infectivity (%) of the viruses. The infectivity (%) is the strength of infectivity in the case where the infectivity of the viruses at 10 degrees C. is 100. The graph of FIG. 7 is a graph of the case where the viruses are influenza viruses. It should be noted that the “temperature of the microorganisms” in FIGS. 6 and 7 is the “indoor temperature of an experimental laboratory at which a microorganism activity control experiment was conducted in the experimental laboratory”.

As illustrated in FIG. 6, as the temperature drops, the generation time (min) in the bacteria shortens and the proliferation rate of the bacteria decreases. That is, the lower the temperature, the less active the bacteria become. By contrast, as illustrated in FIG. 7, as the higher the temperature, the less infective the viruses become. That is, the higher the temperature, the less active the viruses become.

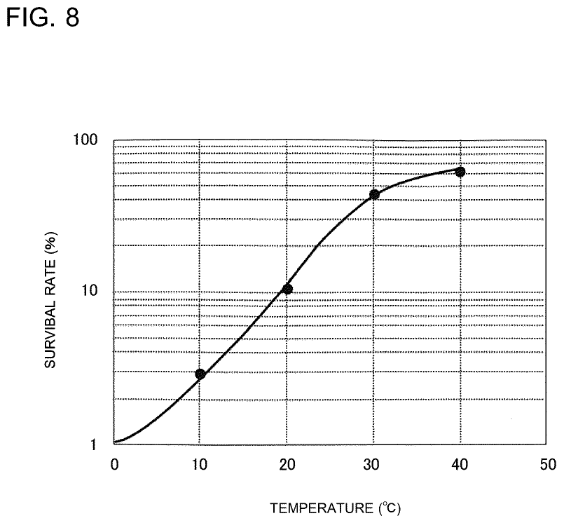

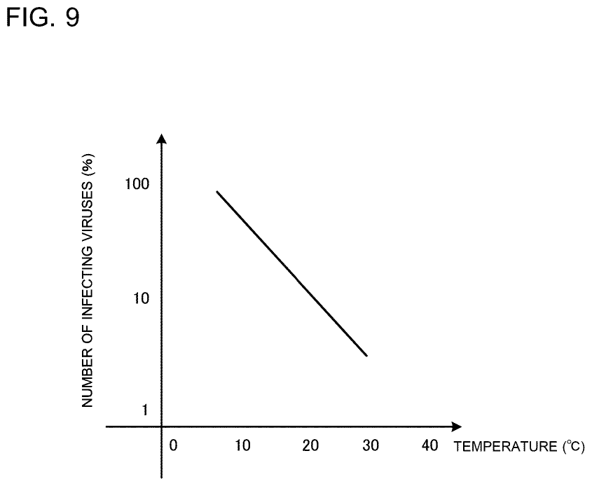

FIG. 8 is a graph indicating an effect of the temperature in the case where bacteria were inactivated by ions. In FIG. 8, the horizontal axis represents the temperature (degree C.) of the microorganisms, and the vertical axis represents the survival rate (%) of the bacteria. The graph of FIG. 8 is a graph of the case where the bacteria are Escherichia coli bacteria. FIG. 9 is a graph indicating an effect of the temperature in the case where viruses were inactivated by ions. In FIG. 9, the horizontal axis represents the temperature (degree C.) of the microorganisms, and the vertical axis represents the remaining number of infecting viruses of viruses (%). The graph of FIG. 9 is a graph of the case where the viruses are influenza viruses. It should be noted that the “temperature of the microorganisms” in FIGS. 8 and 9 is the “indoor temperature of an experimental laboratory at which a microorganism activity control experiment was conducted in the experimental laboratory”.

As illustrated in FIG. 8, the lower the temperature, the lower the survival rate of the bacteria and the greater the disinfection effect by the ions. By contrast, as illustrated in FIG. 9, the higher the temperature, the smaller the remaining number of viruses, and the greater the virus inactivation effect by the ions.

Thus, there is a correlation between the activity of the microorganisms and the temperature of the microorganisms. It can be therefore seen that by reducing the activity of the microorganisms by controlling the temperature of the microorganisms or in this example, the temperature of the air current, it is possible to improve the disinfection and virus inactivation effect by the specific substance such as ions and also possible to reduce the infectivity.

When the temperature of the air flow falls below 25 degrees C., the lower the temperature of the air flow, the greater the disinfection effect. However, when the temperature is lowered too much, cold air is sent to the target space S and this may cause discomfort to a person or persons who are present in the target space S, and increase energy consumption by consuming more energy for cooling. Therefore, the optimal control temperature for execution of the virus inactivation treatment falls within the range of 20 to 25 degrees C.

By contrast, when the temperature of the air flow becomes higher than 28 degrees C., the higher the temperature of the air flow, and the greater the virus inactivating effect. However, when the temperature is too high, warm air is sent to the target space S and this may cause discomfort to a person or persons who are present in the target space S, and increase energy consumption by consuming more energy for heating. Therefore, the optimal control temperature for execution of the virus inactivation treatment falls within the range of 28 to 30 degrees C.

As described above, the disinfection and virus inactivation device 1 adjusts and sets the temperature of the air flow to a temperature at which the activity of the microorganisms to be subjected to disinfection and viral inactivation can decrease, and supplies to the target space, the specific substance along with the air flow the temperature of which is adjusted, thereby improving the disinfection and virus inactivation effect with the specific substance.

As described above, the temperature at which the activity decreases varies depending on the kind of the microorganisms. Therefore, the disinfection and virus inactivation device 1 changes the temperature of the air flow depending on whether the microorganisms are bacteria or viruses. Specifically, in the case of performing the disinfection treatment, the disinfection and virus inactivation device 1 perform a control to set the temperature of the air flow to a first temperature set in advance, and in the case of performing the virus inactivating treatment, the disinfection and virus inactivation device 1 perform a control to set the temperature of the air flow to a second temperature set in advance. The first temperature ranges from 20 to 25 degrees C., and the second temperature ranges from 28 to 30 degrees C.

Switching between the disinfection treatment and the virus inactivating treatment is performed by switching between the treatment modes at the microorganism-activity control unit 21. To be more specific, in the disinfection mode, the microorganism-activity control unit 21 controls the heat source unit 13a to cause the temperature of the air flow to reach the first temperature set in advance, and in the virus inactivating mode, the microorganism-activity control unit 21 controls the heat source unit 13a to cause the temperature of the air flow to reach the second temperature set in advance.

In such a manner, since the microorganism-activity control unit 21 has the disinfection mode and the virus inactivating mode, the disinfection and virus inactivation device 1 can perform the disinfection and virus inactivation operation based on the microorganisms to be treated. Thus, the disinfection and virus inactivation device 1 can maximize the disinfection and virus inactivation effect and shorten the treatment time required for treating the microorganisms.

It should be noted that the temperature at which the activity decreases is the temperature at which the activity of the target microorganisms decreases, and refers to the temperature of an area around the target microorganisms. Therefore, it is preferable that the first temperature and the second temperature be also set in consideration of the temperature in the room.

### Description of Operation

The disinfection and virus inactivation device 1 performs the disinfection and virus inactivation operation in a treatment mode selected by the treatment-mode selecting unit 22. It should be noted that determination of the treatment mode is made by the user. The user inputs the determined treatment mode using the treatment-mode inputting unit 7. In the case where the user wants to perform the disinfection treatment on the target space S, the user inputs a selection of the disinfection mode, and in the case where the user wants to perform the virus inactivating treatment on the target space S, the user does input for section of the virus inactivating mode. The disinfection and virus inactivation device 1 receives, through the treatment-mode reception unit 6, the treatment mode input from the treatment-mode inputting unit 7 and selects, through the treatment-mode selecting unit 22, the received treatment mode.

Then, when receiving a start command to start the disinfection and virus inactivation operation, the disinfection and virus inactivation device 1 starts the disinfection and virus inactivation operation in the treatment mode selected by the treatment-mode selecting unit 22.

The disinfection and virus inactivation device 1 performs the following control in common in the disinfection and virus inactivation operation regardless of whether the treatment mode is the disinfection mode or the virus inactivating mode. That is, the controller 20 of the disinfection and virus inactivation device 1 drives the substance generation unit 10 to generate a specific substance and causes the air-sending device 11a to operate to generate an air flow.

Then, the disinfection and virus inactivation device 1 performs the following control according to the treatment mode. The microorganism-activity control unit 21 controls the activity reducing unit 13 in the treatment mode selected by the treatment-mode selecting unit 22. Specifically, in the case where the treatment mode is the disinfection mode, the microorganism-activity control unit 21 controls the heat source unit 13a to cause the temperature detected by the temperature sensor 13b to the first temperature. By contrast, in the case where the treatment mode is the virus disinfection mode, the microorganism-activity control unit 21 controls the heat source unit 13a to cause the temperature detected by the temperature sensor 13b to reach the second temperature.

By the above control, the specific substance generated by the substance generation unit 10 is supplied together with the air flow the temperature of which is adjusted and set to a temperature suitable for the treatment mode to the target space S through the exhaust outlet 2a1 of the air passage 9a. The air flow that is supplied to the target space S through the exhaust outlet 2a1 and that includes the specific substance reaches and comes into contact with microorganisms in the target space S.

It should be noted that in the case where the disinfection mode is selected, the air flow the temperature of which is adjusted and set to the first temperature which is suitable for reduction of the activity of the bacteria is supplied together with the specific substance to the target space S. Thus, the disinfection and virus inactivation device 1 can treat the bacteria with the specific substance while decreasing the activity of the bacteria by bringing the air flow the temperature of which is adjusted to the first temperature and which contains the specific substance into contact with the bacteria. It should be noted that in Embodiment 1, ions are used as the specific substance, and in the disinfection mode, the bacteria are subjected to the disinfection treatment (hereinafter referred to as “ionic treatment”) with ions contained in the air flow the temperature of which is adjusted to the first temperature.

By contrast, in the case where the virus inactivating mode is selected, the air flow the temperature of which is adjusted and set to the second temperature which is suitable for the reduction of the activity of the viruses is supplied together with the specific substance to the target space S. Thus, the disinfection and virus inactivation device 1 can treat the viruses with the specific substance while decreasing the activity of the viruses by bringing the air flow the temperature of which is adjusted to the second temperature and which contains the specific substance into contact with the viruses. It should be noted that in Embodiment 1, ions are used as the specific substance, and in the virus inactivating mode, the viruses are subjected to the virus inactivating treatment (hereinafter referred to as “ionic treatment”) with ions contained in the air flow the temperature of which is adjusted to the second temperature.

Thus, the disinfection and virus inactivation device 1 can treat the microorganisms with the specific substance while decreasing the activity of the microorganisms to make it easy to perform disinfection, that is, to remove the bacteria, or inactivate the viruses, whereby it is possible to treat the microorganisms efficiently, that is, in a short time period.

Incidentally, during the disinfection and virus inactivation operation, the substance measurement unit 12 measures the specific substance generated from the substance generation unit 10. The substance measurement unit 12 detects the presence or absence of the specific substance, and in the case where the specific substance is present, the substance measurement unit 12 measures the concentration of the specific substance. In the case where the controller 20 detects that the concentration of the specific substance that is measured by the substance measurement unit 12 is lower than or equal to a set concentration set in advance, the controller 20 causes the substance generation unit 10 to stop the operation thereof, and causes the indication unit 5 to operate in a lighting state that indicates a lack of generation of the specific substance. With this, the disinfection and virus inactivation device 1 can make a notification indicating occurrence of an abnormality.

Furthermore, during the disinfection and virus inactivation operation, the temperature sensor 13b measures the temperature of the air flow. In the case where the controller 20 detects that the temperature measured by the temperature sensor 13b deviates from a temperature that is controlled depending on the treatment mode, that is, the first temperature or the second temperature, the controller 20 causes the heat source unit 13a to stop the operation thereof, and causes the indication unit 5 to operate in a lighting state indicating occurrence of a failure in temperature control. With this, the disinfection and virus inactivation device 1 can make a notification indicating occurrence of an abnormality.

### Description of Control Flowchart

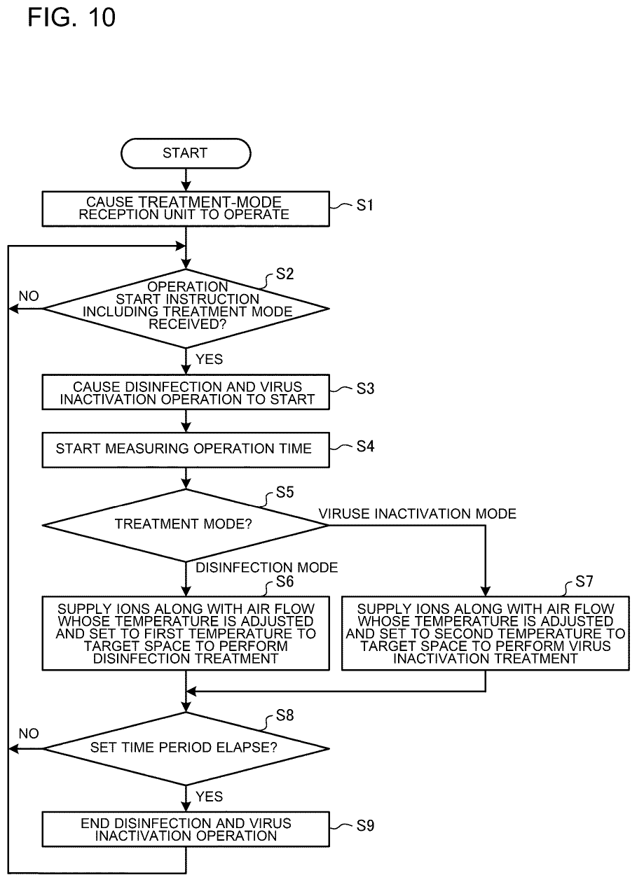

FIG. 10 is a control flowchart of the disinfection and virus inactivation device 1 according to Embodiment 1. The flow of steps of treatment of disinfecting the target space S to remove bacteria or of inactivating viruses in the target space S will be described with reference to the control flowchart of FIG. 10.

When a remote switch (not illustrated) set in the target space S is operated to turn on the power supply of the disinfection and virus inactivation device 1, the controller 20 is activated to cause the treatment-mode reception unit 6 to operate (step S1). When the power supply is in the on-state, the treatment-mode reception unit 6 is in operation at all times and can thus receive, at all times, a treatment mode transmitted from the treatment-mode inputting unit 7. When receiving an operation start instruction including the treatment mode transmitted form the treatment-mode inputting unit 7 (step S2), the controller 20 cause the disinfection and virus inactivation operation to start (step S3). To be more specific, the controller 20 causes the treatment-mode selecting unit 22 to select the treatment mode received by the treatment-mode reception unit 6, and causes the disinfection and virus inactivation operation to start in the selected treatment mode. Furthermore, the controller 20 starts measuring an operation time at the same time as the disinfection and virus inactivation operation starts (step S4).

In the disinfection and virus inactivation operation, when the treatment mode selected by the treatment-mode selecting unit 22 is the disinfection mode (step S5: the disinfection mode), ions generated by the substance generation unit 10 are supplied along with an air flow whose temperature is adjusted and set to the first temperature to the target space S and the disinfection treatment is performed for the bacteria (step S6). Furthermore, in the disinfection and virus inactivation operation, when the treatment mode selected by the treatment-mode selecting unit 22 is the virus inactivating mode (step S5: the virus inactivating mode), ions generated by the substance generation unit 10 are supplied along with an air flow whose temperature is adjusted and set to the second temperature to the target space S and the virus inactivation treatment is performed for the viruses (step S7).

The disinfection and virus inactivation operation is performed for a pet time period set in advance. The disinfection and virus inactivation device 1 starts measuring the operation time at the same time as the disinfection and virus inactivation operation starts, and ends the disinfection and virus inactivation operation (step S9) when the predetermined time period elapses (step S8), and the step to be carried out returns to step S2.

### Advantages

As described above, the disinfection and virus inactivation device 1 according to Embodiment 1 includes the substance generation unit 10 configured to generate a specific substance to perform the disinfection treatment or the inactivating treatment, and the supply unit 11 configured to generate an air flow and supply the specific substance generated from the substance generation unit 10 into the target space. The disinfection and virus inactivation device 1 according to Embodiment 1 further includes the activity reducing unit 13 configured to reduce the activity of microorganisms. Moreover, the disinfection and virus inactivation device 1 supplies the specific substance to the target space S with the supply unit 11, while reducing the activity of the microorganism with the activity reducing unit 13.

With the above configuration, the disinfection and virus inactivation device 1 supplies the specific substance to the target space S while reducing the activity of the microorganisms, and can thus efficiently remove the bacteria as disinfection in the target space S or inactivate the viruses in the target space S. That is, the disinfection and virus inactivation device 1 treats the bacteria or the viruses with the specific substance while reducing the activity of the microorganisms, and can thus efficiently perform disinfection or viral inactivation in the target space S at a high speed.

The disinfection or viral inactivation in the target space S is carried out, for example, during the changeover of people in a meeting room or a satellite office. The disinfection and virus inactivation device 1 can efficiently perform the disinfection treatment or the virus inactivating treatment of microorganisms that float in the target space S, thereby enabling the meeting room or the satellite office to be used next at an earlier timing. Therefore, the disinfection and virus inactivation device 1 can shorten the turnover time for people in the meeting room or the satellite office, increase the utilization rate of the meeting room or the satellite office, and contribute to improvement in work efficiency or expanding the revenue of the satellite office.

The activity reducing unit 13 includes the heat source unit 13a configured to control the temperature of the microorganisms. The heat source unit 13a includes the heating section 13a1 and the cooling section 13a2.

With the foregoing configuration, the disinfection and virus inactivation device 1 can reduce the activity of the microorganisms by controlling the temperature of the microorganisms with the heat source unit 13a. The control of the temperature of the microorganisms can be performed, specifically, by the heating section 13a1 and the cooling section 13a2.

The disinfection and virus inactivation device 1 further includes the microorganism-activity control unit 21 configured to control the heat source unit 13a of the activity reducing unit 13 to cause the temperature of the microorganisms to reach a temperature at which the activity of the microorganisms decreases.

With the foregoing configuration, the disinfection and virus inactivation device 1 can perform a control of causing the temperature of the microorganisms to reach the temperature at which the activity of the microorganisms decreases.

The microorganism-activity control unit 21 has the disinfection mode and the virus inactivating mode. In the disinfection mode, the microorganism-activity control unit 21 performs a control of setting the temperature of the microorganisms to the first temperature set in advance, and in the virus inactivating mode, the microorganism-activity control unit 21 performs a control of setting the temperature of the microorganisms to the second temperature set in advance. The first temperature ranges from 20 to 25 degrees C., and the second temperature ranges from 28 to 30 degrees C.

With the foregoing configuration, the disinfection and virus inactivation device 1 uses the disinfection mode and the virus inactivating mode to perform control of setting the temperature of the microorganisms to a temperature that is suitable for the microorganisms to be treated in the target space S. Thus, the disinfection and virus inactivation device 1 can efficiently remove the bacteria in the target space S as the disinfection or inactivate the viruses in the target space S. Specifically, the disinfection and virus inactivation device 1 can reduce the activity of the microorganisms by setting the temperature of the microorganisms at 20 to 25 degrees C. in the disinfection mode or setting the temperature of the microorganisms at 28 to 30 degrees C. in the virus inactivating mode.

The heat source unit 13a is provided in a flow passage of the air flow and controls the temperature of the microorganisms by controlling the temperature of the air flow.

With the above configuration, the disinfection and virus inactivation device 1 can control the temperature of the microorganisms with the temperature of the air flow.

The disinfection and virus inactivation device 1 further includes the treatment-mode selecting unit 22 configured to select a mode in which the microorganism-activity control unit 21 performs treatment. The microorganism-activity control unit 21 performs treatment associated with the treatment mode selected by the treatment-mode selecting unit 22.

With the above configuration, the disinfection and virus inactivation device 1 can select the treatment mode with the treatment-mode selecting unit 22 and can therefore change the treatment mode on the basis of the microorganisms to be treated.

The treatment-mode selecting unit 22 selects the disinfection mode or the virus inactivating mode in response to an input operation by the user.

With the above configuration, the disinfection and virus inactivation device 1 allows the user to select any of treatment modes.

### Modifications

It should be noted that the control and configuration of the disinfection and virus inactivation device 1 according to the present disclosure are not limited to the control and configuration described above, but the disinfection and virus inactivation device 1 can be modified and put to practical use, for example, in the following manner without departing from the gist of the present disclosure.

Although it is described above that a person determines a treatment mode and inputs the treatment mode to the treatment-mode selecting unit 22, the treatment-mode selecting unit 22 may be modified as descried as follows regarding Modifications 1 to 3.

### Modification 1 of Treatment-Mode Selecting Unit 22

The numbers of bacteria or viruses in a room may be measured, and the treatment-mode selecting unit 22 may automatically determine one of the treatment modes as a treatment mode in which a larger number of bacteria or viruses are to be treated, on the basis of the result of the above measurement.

With the above configuration, the disinfection and virus inactivation device 1 can perform the disinfection and virus inactivation operation without the need for the user to input a treatment mode. Thus, the disinfection and virus inactivation device 1 can reduce the time and effort required for the user's input operation, making the device easier and simpler to use.

### Modification 2 of Treatment-Mode Selecting Unit 22

The treatment-mode selecting unit 22 may be configured to select the disinfection mode or the virus inactivating mode based on the season, an indoor environment, the place (for example, “country”), or a combination thereof. In Japan, bacteria proliferate and tend to cause infection in summer, and viruses tend to cause infection in winter. Specifically, bacteria such as campylobacter, Staphylococcus aureus, or E. coli bacteria start to proliferate around April to May. Thus, the risk that people may be infected with those bacteria is at its peak from July to October. Furthermore, people are easily infected with viruses such as influenza viruses or noroviruses from November to February. Therefore, it is effective that the device operates in the disinfection mode from April to October and operates in the virus inactivating mode from November to March.

For the above purpose, the treatment-mode selecting unit 22 includes a storage unit that stores setting information in which treatment modes are set, for example, on a monthly basis. When the power supply is turned on, the treatment-mode selecting unit 22 may select a treatment mode suitable for the present month based on the storage unit.

With the above configuration, the disinfection and virus inactivation device 1 can perform the disinfection and virus inactivation operation without the need for the user to input a treatment mode. Thus, the disinfection and virus inactivation device 1 can reduce the time and effort required for the user's input operation, making the device easier and simpler to use.

### Modification 3 of Treatment-Mode Selecting Unit 22

Although the treatment-mode selecting unit 22 is configured to select the disinfection mode or the virus inactivating mode during the disinfection and virus inactivation operation, the treatment-mode selecting unit 22 may alternately select the disinfection mode and the virus inactivating mode. Furthermore, after selecting a treatment mode input from the treatment-mode inputting unit 7, the treatment-mode selecting unit 22 may apply the treatment mode that is not input, only when no person is present in the target space S. In these cases, the disinfection and virus inactivation device 1 alternately perform operations associated with the disinfection mode and the virus inactivating mode during the disinfection and virus inactivation operation. As a result, the disinfection and virus inactivation device 1 can treat both the bacteria and the viruses with the disinfection and virus inactivation operation.

Furthermore, although it is described above with respect to the above case that the microorganism-activity control unit 21 has two modes, that is, the disinfection mode and the virus inactivating mode, the microorganism-activity control unit 21 may further have another mode in addition to the above modes. In this case, the treatment-mode selecting unit 22 may select the plurality of treatment modes in turn.

### Modification of Air-Sending Device 11a

In Embodiment 1, as an example, a propeller fan is adopted as the fan of the air-sending device 11a, but a sirocco fan may be adopted. The sirocco fan can send a large volume of air, whereby it is possible to effectively remove the bacteria as disinfection or inactivate the viruses.

### Embodiment 2

Embodiment 2 is intended to supply the specific substance while targeting it on a movement locus of a moving body in the target space S. The following description is made by referring mainly to the differences between Embodiments 1 and 2, and components that will not be described regarding Embodiment 2 are the same as those in Embodiment 1.

Bacteria or viruses adhere to the equipment J in the target space S when a person contacts the equipment J or droplets from a person drop on the equipment J. It has been confirmed that bacteria or viruses adhering to the equipment J retain activity for at least twice as long as bacteria or viruses that are present in the air (Naohide SHINOHARA, Reference Researches on Indoor Environment Valuable for Infection Control of Coronavirus (First Edition), Society of Indoor Environment, Japan (2020)). For this reason, a technique for preventing contact infection, or more specifically, a technique for removing bacteria adhering to the equipment J or other objects in the room or inactivating viruses adhering to the equipment J or other objects in the room, is effective in reducing the risk of infection with bacteria or viruses.

The disinfection and virus inactivation device 1 according to Embodiment 1 described above is configured to supply the specific substance into the target space S in a so-called spraying manner with air sent from the air-sending device 11a of the supply unit 11. For this reason, the disinfection and virus inactivation device 1 according to Embodiment 1 can mainly treat microorganisms floating in the target space S and usage of the disinfection and virus inactivation device 1 according to Embodiment 1 is effective as countermeasures against airborne infection. On the other hand, the disinfection and virus inactivation device 1A according to Embodiment 2 includes a supply unit 11A configured to supply the specific substance toward part of an object with which the moving body in the target space S comes into contact. With this, the disinfection and virus inactivation device 1A according to Embodiment 2 can treat microorganisms adhering to the object touched by the moving body and usage of the disinfection and virus inactivation device 1A according to Embodiment 2 is effective in as countermeasures against contact infection.

The disinfection and virus inactivation device 1A according to Embodiment 2 is different in control from the disinfection and virus inactivation device 1 according to Embodiment 1 on the following two points. One of the points is that the disinfection and virus inactivation device 1A additionally performs a locus detecting operation of detecting a movement locus of the moving body as the locations where the moving body comes into contact with different objects or different portions of an object in the target space S. The other point is that the disinfection and virus inactivation device 1A supplies the specific substance while targeting it on the movement locus of the moving body during the disinfection and virus inactivation operation. In performing the above control, the disinfection and virus inactivation device 1A is also different in structure from the disinfection and virus inactivation device 1 according to Embodiment 1. The following description is made by referring mainly to the differences in control and structure between Embodiments 1 and 2.

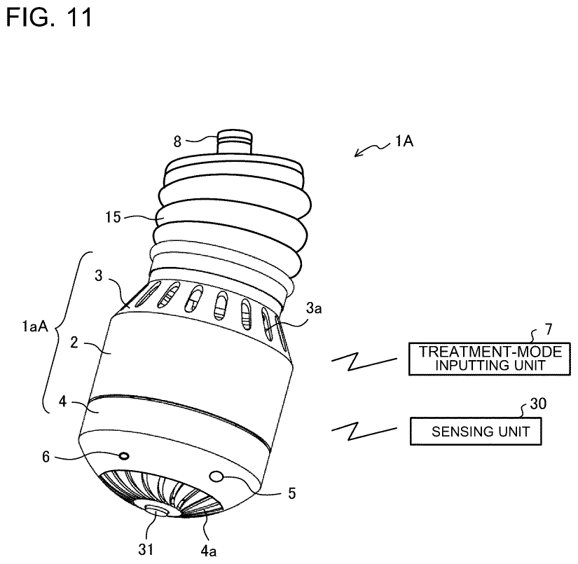

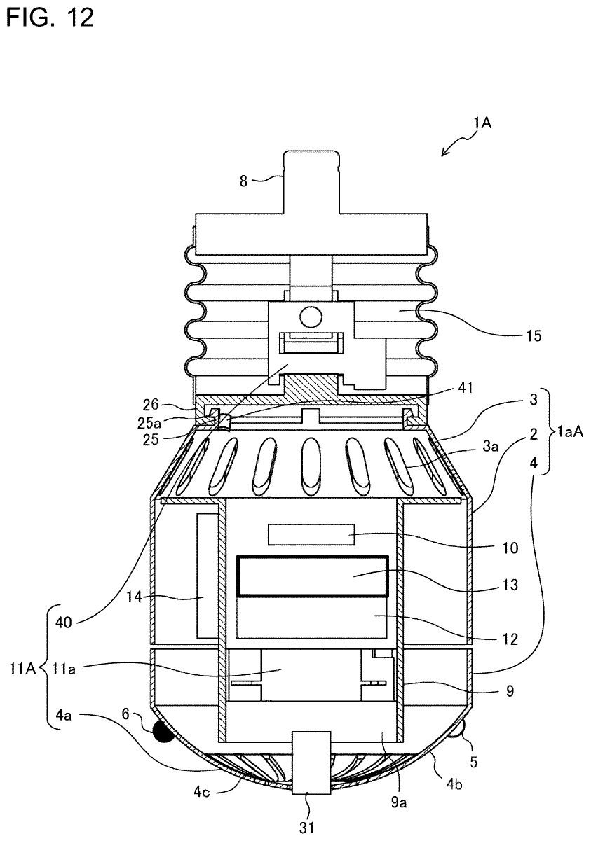

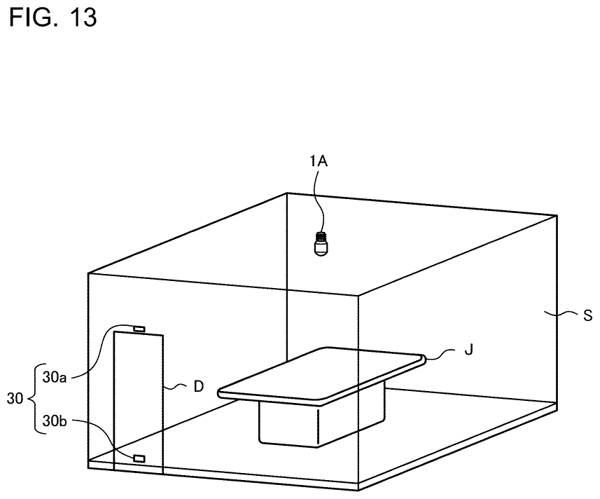

FIG. 11 is an appearance diagram of the disinfection and virus inactivation device 1A according to Embodiment 2. FIG. 12 illustrates an example of a schematic section of the disinfection and virus inactivation device 1A according to Embodiment 2. FIG. 13 illustrates a utilization form of the disinfection and virus inactivation device 1A according to Embodiment 2. The disinfection and virus inactivation device 1A according to Embodiment 2 is different in structure of a housing 1aA from the disinfection and virus inactivation device 1 according to Embodiment 1. To be more specific, the housing 1aA according to Embodiment 2 includes a circular cylindrical tubular portion 2, an annular upper surface portion 3 covering an upper-end opening of the tubular portion 2, and a grille body 4 detachably attached to a lower part of the tubular portion 2.

The grille body 4 is located on a central axis of the air passage 9a. Although not illustrated in detail, the grille body 4 is supported on the inner wall of the tubular portion 2. The grille body 4 includes a grille 4a as a lower part thereof. The grille 4a is a portion that forms part of the supply unit 11A, and will be described later.

Furthermore, the disinfection and virus inactivation device 1A has a bellows portion 15 attached to an upper part of the housing 1aA. The bellows portion 15 is a portion for changing the flow direction of an air flow that is sent out from the grille body 4 and is formed by a bellows-shaped component having flexibility. FIG. 11 illustrates a state in which the flow direction of an air flow sent out from the grille body 4 is changed from a vertically downward direction to an oblique direction.

In the disinfection and virus inactivation device 1A, the bellows portion 15 is attached to the upper part of the housing 1aA, and the grille body 4 is attached to the lower part of the tubular portion 2. Thus, the ferrule 8 is attached to an upper end part of the bellows portion 15. Furthermore, the treatment-mode reception unit 6 and the indication unit 5 are attached to an outer wall of the grille body 4.

The upper surface portion 3 of the housing 1aA is provided with a connector 25 for connection of the housing 1aA to the bellows portion 15. The connector 25 forms part of the housing 1aA. The housing 1aA is detachably attached to the bellows portion 15 when a hook part 25a of the connector 25 is engaged with a locking part 26 provided at a lower end portion of the bellows portion 15. Moreover, in the connector 25, the mode change switch 41 (which will be described later) is provided.

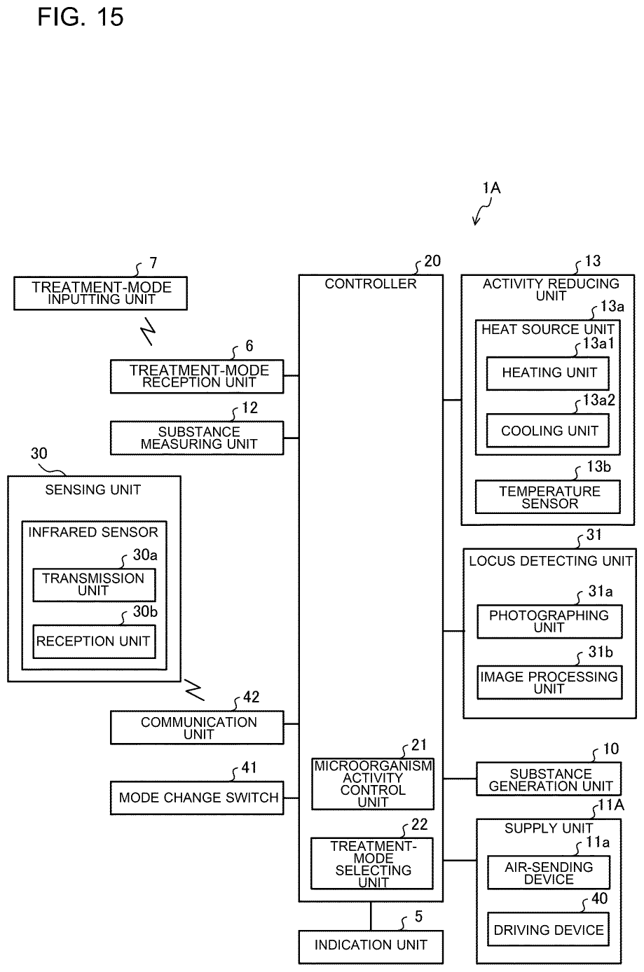

The disinfection and virus inactivation device 1A further includes a sensing unit 30 that is connected to the communication unit 42 (see FIG. 15) in the housing 1aA such that the sensing unit 30 is allowed to communicate with the communication unit 42. The sensing unit 30 is a unit configured to detect the entry of the moving body into the target space S and the exit of the moving body from the target space S (hereinafter referred to as “entry” and “exit”). The sensing unit 30 is provided separately from the housing 1aA. The sensing unit 30 will be described later.

In the housing 1aA, the substance generation unit 10, the supply unit 11A, the substance measurement unit 12, the activity reducing unit 13, the main substrate 14, and a locus detection unit 31 are provided.

The following description is made with respect to components of the disinfection and virus inactivation device 1A that are different from those of the disinfection and virus inactivation device 1.

### Description of Sensing Unit 30

The sensing unit 30 is, for example, an infrared ray sensor. The sensing unit 30 can communicate with the communication unit 42 (which will be described later) provided in the housing 1aA and can send the result of detection of the moving body to the communication unit 42. In this communication, wireless communication such as a wireless LAN, Bluetooth (registered trademark) or ZigBee (registered trademark) is applied. It should be noted that the sensing unit 30 may be a sensing unit already installed in the target space S.

### Description of Locus Detection Unit 31

The locus detection unit 31 detects a movement locus of locations where the moving body comes into contact with different objects or different portions of an object. The locus detection unit 31 is provided in a central part of a lower end of the grille body 4. The moving body to be subjected to locus detection is not only a person but also any biological moving body such as a dog or a cat, as well as a movable appliance such as a movable cleaner. In the following, it is assumed that the moving body is a person unless otherwise described. The configuration and operation of the locus detection unit 31 will be described in detail later.

### Description of Supply Unit 11A

The supply unit 11A generates an air flow that has high straightness and directivity. In addition to the components of the supply unit 11 according to Embodiment 1, the supply unit 11A includes a grille 4a that gives straightness and directivity to the air flow. Furthermore, the supply unit 11A includes a driving device 40 configured to drive the housing 1aA in such a manner as to cause an air flow given straightness and directivity by the grille 4a to be supplied toward the movement locus (which will be described later).

### Grille 4a

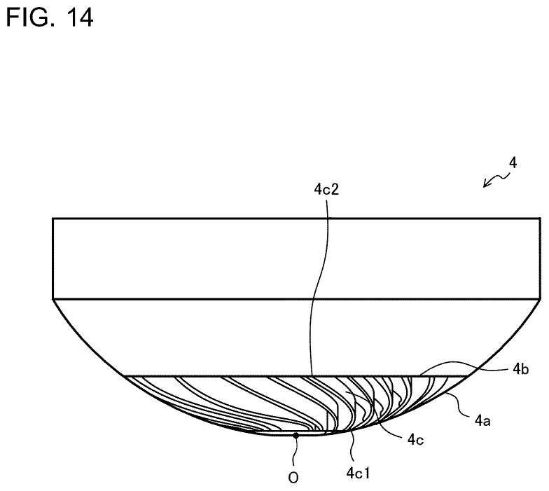

FIG. 14 is a perspective view of the grille body 4 of the disinfection and virus inactivation device 1A according to Embodiment 2. The grille body 4 has an air-sending opening 4b, and the grille 4a is provided at the air-sending opening 4b. The grille 4a has a plurality of spiral fins 4c. The grille 4a is configured such that inner end portions 4c1 of the plurality of fins 4c that are close to a central part O of the spiral further project in an air-sending direction than outer end portions 4c2 of the fins 4c that are continuous with the air-sending opening 4b. In other words, in the grille 4a, the inner end portions 4c1 of the fins 4c further project in the air-sending direction than the outer end portions 4c2 of the fins 4c. The inner end portions 4c1 are located on inner end sides that are close to the central part O of the spiral, and include areas close to inner ends. The outer end portions 4c2 are portions located on outer end sides and formed continuous with the air-sending opening 4b.

With this configuration, the grille 4a can concentrate and converge an air flow that flows out from an outlet of the air passage 9a and flows into the grille body 4 and thereby improve the wind velocity in the center in the air-sending direction. Furthermore, the grille 4a can extend the reaching distance of a spiral air flow that is blown out from the air-sending opening 4b. Thus, the grille 4a can give straightness and directivity to an air flow generated by the air-sending device 11a.

### Driving Device 40

FIGS. 11 and 12 will be re-referred to. In order that an air flow blown out from the grille 4a flow toward a movement locus detected by the locus detection unit 31, the driving device 40 changes the orientation of the grille 4a by driving the housing 1aA and controls the air-sending direction of the air flow. When the housing 1aA is driven by the driving device 40, the bellows portion 15 is deformed to change the air-sending direction. The driving device 40 includes a motor (not illustrated) capable of driving with respect to two shafts orthogonal to each other. The motor is a common servo motor or a stepping motor. These motors can control the angle of a shaft that supports the housing 1aA and cause the shaft to stop at a specified position. Thus, the driving device 40 can cause the grille 4a provided at the air-sending opening 4b to stop accurately while facing toward the movement locus.

By having the above configuration, the supply unit 11A can cause the straightness and directivity of an air flow generated by the air-sending device 11a to be enhanced by the grille 4a and supply the air flow toward the movement locus.

FIG. 15 is a block diagram of the disinfection and virus inactivation device 1A according to Embodiment 2. By lead wires, the locus detection unit 31, the driving device 40, the mode change switch 41, and the communication unit 42 are electrically connected to the controller 20 in addition to the components according to Embodiment 1 as illustrated in FIG. 4. The communication unit 42 has a function of performing wireless communication such as a wireless LAN, Bluetooth (registered trademark) or ZigBee (registered trademark), and performs wireless communication with the sensing unit 30. It should be noted that the communication unit 42 may be provided on the same mounting board as or a different mounting board from both/either the treatment-mode reception unit 6 and/or the indication unit 5. In the case where the communication unit 42 may be provided on the same mounting board as or the different mounting board from both/either the treatment-mode reception unit 6 and/or the indication unit 5, the disinfection and virus inactivation device 1A can be manufactured at a low cost.

The controller 20 controls the locus detection unit 31, the substance generation unit 10, the air-sending device 11a, and the driving device 40 based on the result of detection by the sensing unit regarding entry/exit of a person into/from a room. The controller 20 performs the locus detecting operation in addition to the above disinfection and virus inactivation operation. The locus detecting operation will be described later.

The disinfection and virus inactivation device 1A detects entry/exit of a person by performing wireless communication with the sensing unit 30 through the communication unit 42. Specifically, the disinfection and virus inactivation device 1A acquires, via the communication unit 42, a sensing signal sent from the sensing unit 30 and indicating the entry/exit of a person, and detects the entry/exit of the person from the sensing signal. The disinfection and virus inactivation device 1A detects, from the sensing signal sent from the sensing unit 30, that the first person enters the room and that all people exit the room.

The infrared ray sensor that forms the sensing unit 30 has a transmission unit 30a configured to transmit infrared rays and a reception unit 30b configured to receive infrared rays. The transmission unit 30a and the reception unit 30b are installed close to the door D of the target space S. Specifically, the transmission unit 30a and the reception unit 30b are separately provided above and below an entrance, and infrared rays are transmitted and received between the transmission unit 30a and the reception unit 30b.

When no person passes through the space between the transmission unit 30a and the reception unit 30b, the amount of the infrared rays received by the reception unit 30b does not change and is substantially constant. By contrast, when a person passes through the space between the transmission unit 30a and the reception unit 30b, the amount of infrared rays that are received by the reception unit 30b decreases. When the amount of infrared rays that are received by the reception unit 30b falls below a specified value, the infrared ray sensor detects movement of a person. Furthermore, a method for detecting entry/exit of a person with the infrared ray sensor is simpler and more inexpensive in device configuration than a method for detecting entry/exit of a person with image data. It should be noted that the sensing unit 30 is not limited to the infrared ray sensor but may be any type of devices long as the device is capable of detecting entry/exit of a person.

The sensing unit 30 may be installed close to the door D as described above, or in the case where the target space S is, for example, a restroom, the sensing unit 30 may be installed close to a toilet bowl of the restroom.

The locus detection unit 31 is a unit configured to perform a process of detecting a movement locus of locations where a moving body comes into contact with different objects or different portions of an object. The movement locus is a locus of locations where a person touches different objects or different portions of an object, such as locations where the person's hand touches the different objects or different portions of the object or locations where the person walks. However, in the following description, of the locations where the person touches the different objects or different portions of the single object, locations where the person touches different objects whose infection risks are high or different portions of an object whose infection risks are high are detected as a movement locus. To be more specific, it is assumed that the movement locus is a locus of locations where the person touches different portions of the equipment J.

The locus detection unit 31 includes a photographing unit 31a configured to photograph the target space S and an image processing unit 31b configured to perform locus detection based on photographic data obtained by the photographing unit 31a.

### Photographing Unit 31a

The photographing unit 31a photographs the interior of the target space S. The photographing unit 31a includes an imaging element, a lens unit, a lens holder, and a cover plate. The imaging element includes a solid-state imaging element such as a complementary metal-oxide semiconductor (CMOS) image sensor or a charge-coupled device (CCD) image sensor that can acquire image data. The lens unit is provided in front of the imaging element. The lens unit includes a lens that condenses light and an adjuster unit configured to relatively vary the distance between the imaging element and the lens. The adjuster unit includes a permanent magnet that holds the lens and an electromagnetic coil that renders the permanent magnet movable.

The lens unit renders the lens movable by adjusting an electric current flowing through the coil and adjusts the focus of the lens on the imaging element. The lens holder holds the lens unit. The lens holder has an annular outer shape. The cover plate is provided to close an opening of the annular lens holder. The cover plate is provided in front of the lens unit. The cover plate has translucency. The cover plate is colored such that it becomes hard to view the interior of the photographing unit 31a from the outside.

### Image Processing Unit 31b

The image processing unit 31b includes an arithmetic control unit, a first storage unit, and a second storage unit. The arithmetic control unit performs arithmetic processing on image data obtained by photographing by the photographing unit 31a. The arithmetic control unit includes a field-programmable gate array (FPGA) and a digital signal processor (DSP). Instead of using the DSP, the arithmetic control unit may use a semiconductor element, such as an advanced image processor, which is capable of performing digital image processing at a high speed.

The first storage unit stores image data obtained by photographing by the photographing unit 31 in advance, when no person is present in the target space S. The image data obtained by photographing in advance when no person is present is used as background data at the time of performing a moving-body detection processing of differentiating a human and a non-human from each other. The first storage unit is a nonvolatile memory such as a synchronous DRAM (SDRAM), which enables the image data to be transferred to the arithmetic control unit at a high speed.

The second storage unit stores, as image data, tracking data on a person who is present in the target space S. The second storage unit is a mass-storage device that is capable of storing a large amount of image data. The mass-storage device corresponds, for example, to a volatile memory, such as a dynamic random-access memory (DRAM), whose storage capacity is relatively large.

The image processing unit 31b stores the background data in the first storage unit. The image processing unit 31b reads the background data stored in the first storage unit into the arithmetic control unit as appropriate. The image processing unit 31b reads, into the arithmetic control unit, current image data obtained by photographing that is performed by the photographing unit 31a on a regular basis.

The arithmetic control unit reads the current image data and the background data and performs an image difference processing using the current image data and the background data. The image difference processing is processing in which the current image data is compared with the background data, a difference image is produced by taking a difference for each of pixels, and the produced difference image is then subjected to binarization processing using a threshold set in advance, thereby producing a binarized image. The production of the difference image is not limited to production thereof by background difference processing only, but the difference image may be produced by time difference processing based on comparison between two image data items that are read in different time series by the imaging element.

The luminance value of a difference in each of pixels in which no change is made between the current image data and the background data falls below the threshold set in advance. By contrast, the luminance value of a difference in each of pixels in which a change is made between the current image data and the background data, that is, in each of pixels of a portion in which a person is photographed, exceeds the threshold set in advance. Therefore, the arithmetic control unit extracts the portion in which a person is present by performing, using the threshold, binarization processing on a difference image between the current image data and the background data.

The arithmetic control unit causes a binarized image that is acquired such that the portion in which the person is present is extracted to be stored by the above image processing in the second storage unit as a location in which the person is present. The arithmetic control unit produces a binarized image by performing the image difference process each time image data is output from the photographing unit, and stores the produced binarized image in the second storage unit. The arithmetic control unit can detect, through binarized images stored on a time-series basis in the second storage unit, a movement locus of a person who is present in the target space S. That is, the arithmetic control unit can track the movement of the person by comparing the current binarized image with binarized image produced before a certain time period. While the locus detection unit 31 continues to track the person until the person exits the target space S, the arithmetic control unit continues to store locus detection data as binarized images in the second storage unit.

Furthermore, the arithmetic control unit can recognize the location of the equipment J in advance based on the background data and detect a location where the person comes into contact with the equipment, from a positional relationship between the location of the equipment and the location in which the person is present. Specifically, the arithmetic control unit acquires in advance a difference image between image data on a state in which the equipment J is not installed and image data on a state in which the equipment J is installed, that is, a difference image obtained by extracting the equipment J. Then, the arithmetic control unit detects, as the location where the person comes into contact with the equipment J, an overlap between the difference image obtained by extracting the equipment J and the binarized image obtained by extracting the portion in which the person is present.

With the above configuration, the locus detection unit 31 can detect a movement locus of locations where the person comes into contact with different objects or different portions of an object in the target space S. This movement locus includes a movement locus of locations where the person comes into contact with the ground when the person walks on the ground and a movement locus of locations where the person touches different portions of the equipment J with his/her hand.

### Description of Operation

The disinfection and virus inactivation device 1A performs the locus detecting operation and the disinfection and virus inactivation operation. First of all, the locus detecting operation will be described, and then, the disinfection and virus inactivation operation will be described.

### Locus Detecting Operation

The locus detecting operation is an operation that is performed in the case where a person is present in the target space S, and is an operation of detecting a movement locus of locations where the person comes into contact with different objects or different portions of an object.

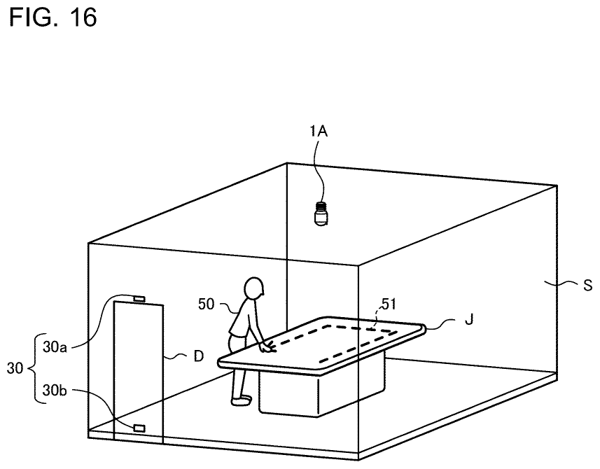

FIG. 16 is an explanatory view for the locus detecting operation of the disinfection and virus inactivation device 1A according to Embodiment 2. In the locus detecting operation, the sensing unit 30 detects entry/exit of a person 50 into/from the target space S. In the locus detecting operation, when the sensing unit 30 detects entry of the person 50 into the target space S, the disinfection and virus inactivation device 1A starts detecting a movement locus of locations where the person 50 comes into contact with different objects or different portions of an object. The disinfection and virus inactivation device 1A continues to detect the movement locus of the locations where the person 50 comes into the different objects or different portions of the single object until the sensing unit 30 detects exit of the person 50 from the target space S. In FIG. 16, dashed arrows indicate a movement locus 51 of the person 50 and also locations where the person 50 comes into contact with different portions of the equipment J.

During the locus detecting operation, the disinfection and virus inactivation device 1A causes the indication unit 5 to light in a lighting manner that indicates that the locus detecting operation is being performed. In such a manner, the disinfection and virus inactivation device 1A can make a notification indicating details of the operation.

### Disinfection and Virus Inactivation Operation

The disinfection and virus inactivation operation is intended to reduce the risk of contact infection by supplying the specific substance toward the movement locus in the target space S to remove bacteria or inactivate viruses. As in Embodiment 1, the disinfection and virus inactivation operation according to Embodiment 2 treats microorganisms in the target space S by supplying the specific substance generated by the substance generation unit 10 to the target space S after reducing the activity of the microorganisms by control of the temperature of the microorganisms. The following description is made by referring to by way of example the case where the temperature of the microorganisms is controlled by controlling the temperature of an air flow as in Embodiment 1.

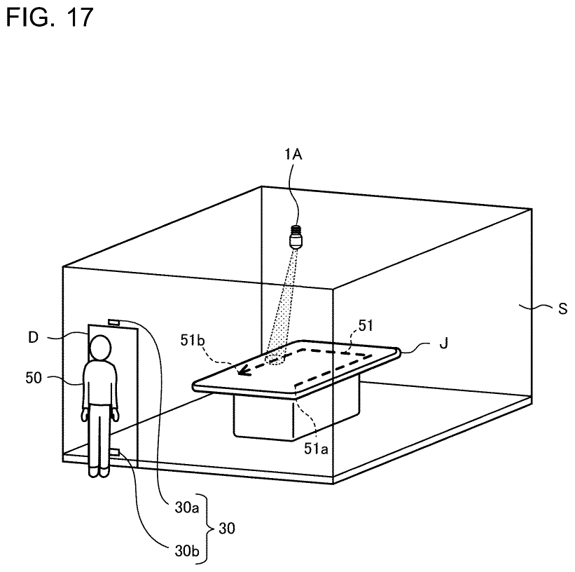

FIG. 17 is an explanatory diagram for the disinfection and virus inactivation operation of the disinfection and virus inactivation device 1A according to Embodiment 2. The disinfection and virus inactivation operation is also an operation that is performed in the case where the person 50 exits the target space S, and is an operation of supplying the specific substance generated by the substance generation unit 10 to the movement locus 51, as illustrated in FIG. 17. The disinfection and virus inactivation operation is performed until the sensing unit 30 detects the entry of the person 50.

When the sensing unit 30 detects the exit of the person 50, the disinfection and virus inactivation device 1A starts the disinfection and virus inactivation operation by driving the substance generation unit 10, the activity reducing unit 13, and the supply unit 11A. Specifically, the controller 20 drives the substance generation unit 10 to generate the specific substance and cause the air-sending device 11a to start operating. Furthermore, the microorganism-activity control unit 21 controls the activity reducing unit 13 in a treatment mode selected by the treatment-mode selecting unit 22. It should be noted that it suffices that the selection of the treatment mode by the treatment-mode selecting unit 22 is performed, for example, when the person 50 is present in the room before the start of the disinfection and virus inactivation operation.

Ions generated as the specific substance by the substance generation unit 10 are carried along with an air flow whose temperature is adjusted and set to a temperature suitable for the treatment mode, toward the outlet of the air passage 91, by the operation of the air-sending device 11a and the activity reducing unit 13. At the same time as the ion are carried, the controller 20 controls the driving device 40 so that the grille 4a of the grille body 4 faces toward the movement locus 51 detected by the locus detection unit 31.

As a result, the specific substance generated by the substance generation unit 10 rides on the air flow having its straightness and directivity enhanced by the grille 4a and having its temperature adjusted and set to a temperature at which the activity of the microorganisms decreases, and is supplied toward the movement locus 51 of the person 50 in the target space S. The disinfection and virus inactivation device 1A controls the orientation of the grille 4a of the grille body 4 to supply the specific substance along the movement locus 51 from a starting point 51a toward an ending point 51b.

It should be noted that the movement locus 51 is a movement locus of a person 50 carrying bacteria or viruses, and corresponds to locations of portions of an object in the target space S to which a larger number of bacteria or viruses adhere. The structure of the present example makes it possible to supply the specific substance toward the movement locus 51 by causing, without causing the specific substance to diffuse into the target space S, the specific substance to ride on the air flow having high straightness and directivity and having its temperature adjusted and set to the temperature at which the activity of the microorganisms decrease. That is, the disinfection and virus inactivation device 1A can send the specific substance toward a place in which a larger number of bacteria or viruses are present. Thus, the disinfection and virus inactivation device 1A can send the specific substance to the movement locus 51, with their concentrations made high, upon controlling and setting of a microorganism ambient temperature to a temperature environment in which the activity of the microorganisms decreases, and can thus efficiently remove bacteria that are present on the movement locus 51 or inactivate viruses that are present on the movement locus 51.

Furthermore, the disinfection and virus inactivation device 1A can focus on the inactivation of bacteria or viruses at the portion or portions touched by the person 50 and can thus reduce the risk of contact infection in the target space S. In addition, the disinfection and virus inactivation device 1A is provided at a high place in the target space S such as a ceiling, and can thus more easily supply the specific substance to a surface portion of the equipment J that is easily touched by the person 50 than in the case where the disinfection and virus inactivation device 1A is provided on a floor surface.

Furthermore, since the temperature of the flow of air is adjusted and set to a temperature suitable for the treatment mode, bacteria or viruses can be treated after the activity thereof is reduced. It is therefore possible to treat bacteria or viruses in a short time period.

### Description of Operation

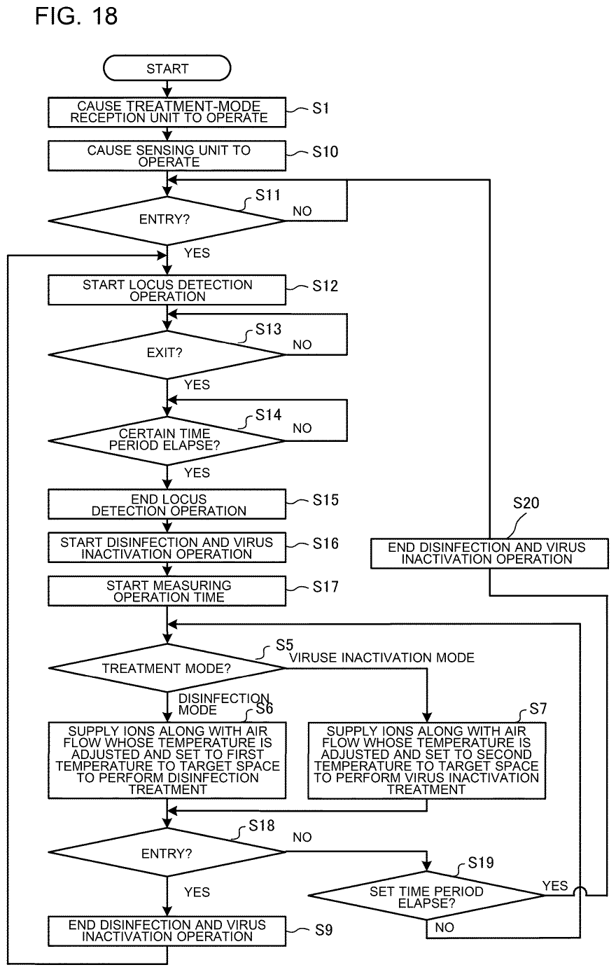

FIG. 18 is a control flowchart of the disinfection and virus inactivation device 1A according to Embodiment 2. In FIG. 18, steps that are the same as those in FIG. 10 that is the control flowchart according to Embodiment 1 are denoted by the same step numbers. It will be described with reference to the flowchart of FIG. 18 how the disinfection and virus inactivation device 1A operates. The following description is made by referring mainly to the differences between Embodiments 1 and 2.

When a remote switch (not illustrated) set in the target space S is operated to turn on the power supply of the disinfection and virus inactivation device 1A, the controller 20 is activated to cause the treatment-mode reception unit 6 to operate (step S1). When the power supply is in the on-state, the treatment-mode reception unit 6 is in operation at all times and can thus receive at all times, a treatment mode transmitted from the treatment-mode inputting unit 7. Furthermore, when the power supply is turned on, the disinfection and virus inactivation device 1A causes the sensing unit 30 to operate (step S10). When the power supply is in the on-state, the sensing unit 30 is in operation at all times to detect entry/exit of the person 50 into/from the target space S. The controller 20 detects entry/exit into/from the target space A at all times from a sensing signal from the sensing unit 30 (No in step S11). When detecting entry of the person 50 (Yes in step S11), the controller 20 causes the locus detection unit 31 to operate and start the locus detection operation (step S12). The locus detection unit 31 continues to detect a movement locus of the person 50 until the sensing unit 30 detects the exit of the person 50 (No in step S13).

When detecting the exit of the person 50 from the target space S from the sensing signal from the sensing unit 30 (Yes in step S13), the controller 20 checks whether a certain time period elapses or not (step S14). When the certain time period does not elapse (No in step S14), the controller 20 continues the locus detection operation. By contrast, when the certain period elapses (Yes in step S14), the controller 20 ends the locus detection operation (step S15) and starts the disinfection and virus inactivation operation (step S16). That is, the controller 20 switches the operation from the locus detection operation to the disinfection and virus inactivation operation. Then, the controller 20 starts measuring the operation time at the same as the disinfection and virus inactivation operation starts (step S17).

In the disinfection and virus inactivation operation, as described above, the controller 20 drives the substance generation unit 10 to cause the substance generation unit 10 to generate a specific substance and also causes the air-sending device 11a to start operating. In addition, in the disinfection and virus inactivation operation, the controller 20 further causes the activity reducing unit 13 to operate in the operation mode selected by the treatment-mode selecting unit 22 (steps S5, S6, and S7).

The disinfection and virus inactivation operation is performed for the set time period set in advance. However, in the case where the person 50 enters the target space S before the set time period elapses, the disinfection and virus inactivation operation is stopped. To be more specific, the controller 20 performs the following processing.

When the entry of the person 50 is not detected after the start of the disinfection and virus inactivation operation (No in step S18), the controller 20 checks whether the set time period elapses from time at which the controller 20 starts measuring operation time or not (step S19). In the case where the set time period does not elapse (No in step S19), the processing by the controller 20 returns to step S5 and the controller 20 repeats the processes of steps S5, S6, S7, S18, and S19. Then, in the case where the controller 20 determines that the set time period elapses without detection of the entry of the person 50 (Yes in step S19), the controller 20 ends the disinfection and virus inactivation operation (step S20) and the processing by the controller 20 returns to step S11.

Furthermore, when the entry of the person 50 is detected in step S18 during the disinfection and virus inactivation operation (Yes in step S18), the controller 20 ends the disinfection and virus inactivation operation (step S9). That is, the controller 20 stops driving of the substance generation unit 10 to stop generation of the specific substance, and stops the operation of the air-sending device 11a to stop the operation of the activity reducing unit 13. After the stoppage of the disinfection and virus inactivation operation, the processing by the controller 20 returns to step S12 and the controller 20 restarts the locus detecting operation.

The controller 20 is configured to switch the operation from the locus detecting operation to the disinfection and virus inactivation operation after a certain time period elapses from the time at which the exit of the person 50 from the target space S is detected, for the following reason. In the case where the operation is switched from the locus detecting operation to the disinfection and virus inactivation operation immediately after the exit of the person 50 is detected, if a larger number of persons enter and exit the target space S, the switching of the operation is frequently performed. For this reason, the controller 20 switches the operation from the locus detecting operation to the disinfection and virus inactivation operation with a time lag of a certain time period. As a result, the disinfection and virus inactivation device 1A can reduce the number of times the switching of the operation is performed, and can reduce the load on the air-sending device 11a.

The disinfection and virus inactivation device 1A includes the mode change switch 41 for use in setting the time lag of the certain time period. The mode change switch 41 is a slide switch, is provided in the connector 25, and is not visible from the outside. By detaching the bellows portion 15 from the housing 1aA, the user can operate the mode change switch 41 through an opening formed in the upper part of the housing 1aA. It should be noted that the certain time period here is a time period determined in advance in an initial state. The user can change the certain time period by changing a slide position of the mode change switch 41. For example, at a first slide position, the certain time period is set to 30 seconds, and at a second slide position, the certain time period is set to 1 minute. It should be noted that the mode change switch 41 is not limited to the slide switch.

As described above, the disinfection and virus inactivation device 1A performs the disinfection and virus inactivation operation when the person 50 exits the target space S and the person 50 is not present in the target space S. Thus, no problem arises even when a temperature control of the activity reducing unit 13 greatly changes the temperature as compared with Embodiment 1. Specifically, in view of, for example, power consumption, an optimum temperature control may be performed in the following manner. Since the disinfection effect is obtained at 20 to 25 degree C. and the virus inactivating effect is obtained at 28 to 30 degrees C., the disinfection and virus inactivation device 1A may be in temperature set to 20 degrees C. to enhance the disinfection effect in summer and set to 30 degrees C. to enhance the virus inactivating effect. Thus, the disinfection and virus inactivation device 1A can also be in temperature set to a temperature that greatly differs from the temperature in an outside air environment and at which a person may feel discomfort. This allows the disinfection and virus inactivation device 1A to effectively remove bacteria or inactive viruses.

Although the forgoing description is made with respect to the case where only one person 50 enters the target space, a plurality of persons 50 may enter the target space. In this case, the disinfection and virus inactivation device 1A performs the locus detecting operation until everyone exits the target space after the first person enters the target space, and performs the disinfection and virus inactivation operation after everyone exits the target space.

In Embodiment 1, ions are used as the specific substance. Ions are known as a substance having low residual properties. In the case where the specific substance is such a low residual substance, even if the concentration of the specific substance in the target space S increases to a high concentration during the disinfection and virus inactivation operation, the concentration decreases sharply when the disinfection and virus inactivation operation is stopped and driving of the substance generation unit 10 is stopped. For this reason, even if a high concentration of the specific substance is supplied into the target space S from the disinfection and virus inactivation device 1A during the disinfection and virus inactivation operation, the safety of the person 50 who enters the target space S after the disinfection and virus inactivation operation can be ensured. Therefore, in the case where a low residual substance is used as the specific substance, the disinfection and virus inactivation device 1 causes the specific substance to be generated from the substance generation unit 10 such that the concentration of the specific substance in the target space S reaches a high concentration. The high concentration is not limited to a specific numerical value but may be set to a concentration that is effective in promoting the efficiency of disinfection or viral inactivation.

Furthermore, as the specific substance, hypocholorous acid water may be used instead of ions. Ions and hypocholorous acid water differ from each other in the degree of decrease in the disinfection and virus inactivation effect due to temperature. As the temperature rises, and more specifically, when the temperature reaches 28 degrees C. or higher, the disinfection and virus inactivation effect of hypocholorous acid water decreases rapidly. Therefore, it is preferable that the disinfection and virus inactivation device 1A perform a control of causing the temperature of the air flow to fall within a temperature range in which the disinfection and virus inactivation effect of the specific substance does not decrease. That is, it is preferable that the disinfection and virus inactivation device 1A set the temperature of the air flow to 28 degrees C. when applying the virus inactivating mode with hypocholorous acid water. Because of such a setting, the disinfection and virus inactivation device 1A can inactivate viruses with hypocholorous acid water while decreasing the activity of the microorganisms, and as a result can efficiently inactivate the viruses.

During the disinfection and virus inactivation operation, the disinfection and virus inactivation device 1A may first operate in a treatment mode selected by the treatment-mode selecting unit 22 and then in a treatment mode that is not selected. During the disinfection and virus inactivation operation of the disinfection and virus inactivation device 1A, the disinfection mode and the virus inactivating mode may be alternately applied. In these cases, the disinfection and virus inactivation device 1 can treat both the bacteria and the viruses by performing the disinfection and virus inactivation operation.

It should be noted that of specific substances, some specific substances have not been clarified with respect to whether they are safe for the human body or not. However, since the disinfection and virus inactivation device 1A performs the disinfection and virus inactivation operation when no person is present, no person inhales or touches the specific substance. Therefore, the disinfection and virus inactivation device 1A can achieve an effective disinfection and virus inactivation operation without doing harm to the person.

### Advantages

As described above, in addition to the same advantages as obtained by the disinfection and virus inactivation device 1 according to Embodiment 1, the disinfection and virus inactivation device 1A according to Embodiment 2 obtains the following advantages. In addition to the components of the disinfection and virus inactivation device 1, the disinfection and virus inactivation device 1A further includes the locus detection unit 31 configured to detect a movement locus of locations where a moving body comes into contact with different objects or different portions of an object in the target space S. Furthermore, the supply unit 11A of the disinfection and virus inactivation device 1A supplies the specific substance toward the movement locus of the disinfection and virus inactivation device 1A. Thus, with an air flow whose temperature is controlled and set to a temperature at which the activity of the microorganisms decreases, the disinfection and virus inactivation device 1A can supply the specific substance toward a movement locus 51 of locations where the moving body comes into contact with different objects or different portions of an object in the target space S, and more efficiently perform disinfection or inactivate the viruses in the target space S.

The supply unit 11A includes an air-sending device 11a configured to generate an air flow, the grille 4a provided downstream of the air-sending device 11a and configured to give straightness and directivity to the air flow from the air-sending device 11a, and the driving device 40 configured to control the flow direction of the air flow by changing the orientation of the grille 4a. The supply unit 11A supplies the air flow along the movement locus 51 by changing the orientation of the grille 4a with the driving device 40.

Thus, as the air flow that is given straightness and directivity by the grille 4a, the air flow whose temperature is controlled and set to the temperature at which the activity of the microorganisms decreases is supplied along the movement locus 51 by the disinfection and virus inactivation device 1A. Therefore, the disinfection and virus inactivation device 1A can efficiently and intensively disinfect a place whose infection risk is high or inactivate viruses thereat.

The disinfection and virus inactivation device 1A further includes the sensing unit 30 configured to detect exit of the moving body from the target space S. In the case where the sensing unit 30 detects that the moving body exits the target space S, the supply unit 11A supplies the specific substance generated by the substance generation unit 10 toward the movement locus, using the air flow whose temperature is controlled or set to the temperature at which the activity of the microorganisms decreases.

In such a manner, since the disinfection and virus inactivation device 1A starts the disinfection and virus inactivation operation upon detection of exit of the moving body from the target space S, the disinfection and virus inactivation device 1A can efficiently and intensively perform disinfection or viral inactivation while the moving body is not present in the target space S. Furthermore, in other words, since the disinfection and virus inactivation device 1A does not perform the disinfection and virus inactivation operation while the moving body is present in the target space S, the disinfection and virus inactivation device 1A can keep the moving body comfortable in the target space S and effectively perform the disinfection and virus inactivation operation.

When the sensing unit 30 detects that the moving body exits the target space S, the supply unit 11A supplies the specific substance generated by the substance generation unit 10 toward the movement locus after elapse of a certain time period set in advance.

With the above configuration, the disinfection and virus inactivation device 1A can reduce the number of times the switching of the operation is performed in the case where the moving body often enters and exits the target space S, and can reduce the load on the air-sending device 11a. Furthermore, the disinfection and virus inactivation device 1A does not cause discomfort for the moving body in terms of noise and cold sensation, for example, even in the case where the moving body returns to the target space S immediately after exiting the target space S.

### Modification

It should be noted that the configuration of the disinfection and virus inactivation device 1A according to the present disclosure is not limited to such a configuration as described above but the disinfection and virus inactivation device 1A can be modified and put to practical use, for example, in the following manner without departing from the scope of the present disclosure.

### Modification of Configuration

Although it is described above that the sensing unit 30 provided separately from the housing 1aA detects entry/exit of the moving body into/from the target space S, the locus detection unit 31 may be configured to double as the sensing unit 30. In the case where the locus detection unit 31 is configured to double as the sensing unit 30, the locus detection unit 31 executes the following processing to become able to detect entry/exit of the person 50 into/from the target space S as the sensing unit 30 does.

The locus detection unit 31 performs an image difference process for generating a difference image between background data representing the door D in the target space S and image data that is obtained by photographing the door D at the present time. In the difference image obtained by the image difference process, in the case where a difference in luminance value is detected in the image of the door D in the target space, the locus detection unit 31 can detect that the person 51 enters or exits the target space S. Furthermore, the locus detection unit 31 can detect entry/exit of the person 50 into/from the target space S in such a manner as to determine that the person 50 enters the target space S, when the direction of the change in the difference image is a direction toward the inside of the target space S, and determine that the person 50 exits the target space S, when the direction of the change in the difference image is the direction toward the outside of the target space S.

Furthermore, in the case where the locus detection unit 31 is configured to double as the sensing unit 30, the locus detection unit 31 may include a visible-light sensor or a ultrasonic sensor to detect the movement locus 51 of the moving body on the basis of the result of detection by the sensor.

Although it is described above that the target space S is, for example, the closed space partitioned off by the partition, the target space S may be an unclosed space. The unclosed space corresponds, for example, to a space provided by virtually partitioning off part of a large space such as a banquet hall. When the target space S is an unclosed space, the disinfection and virus inactivation device 1A can perform the disinfection and virus inactivation operation, for example, by deeming part of a large space as the target space S without the need to provide a closed spaced by physically partitioning off the large space. In such a manner, in the case where the target space S is an unclosed space, the disinfection and virus inactivation device 1A is installed in a place where the specific substance generated by the substance generation unit 10 can be supplied to the target space S using the supply unit 11A.

It suffices that an imaginary line of demarcation of an unclosed space is set and entry/exit of a moving body into/from the entire area of the line of demarcation is monitored, thereby to detect entry/exit of the moving body into/from the unclosed space. Specifically, it suffices that a plurality of sensing units 30 are provided at respective positions on the imaginary line of demarcation such that it is possible to detect entry/exit of the moving body through the plurality of sensing units 30. When entry of the moving body is detected, the locus detection unit 31 detects a locus of the moving body until the moving body exits the space. Then, the disinfection and virus inactivation device 1A performs the disinfection and virus inactivation operation after confirming the exit of the moving body with the sensing units 30. In such a manner, by providing the sensing units 30 in the entire area of the imaginary line of demarcation, it is possible for the disinfection and virus inactivation device 1A to perform disinfection and viral inactivation on an undivided space.

Although in the above configuration, a coaxial bicylindrical ion sensor is employed as the ion sensor that forms the substance measurement unit 12, a parallel plate type ion sensor may be employed. The parallel plate type is a type in which ions flowing between flat-plate electrodes provided parallel to each other are measured from the amount of current between the flat-plate electrodes. The parallel plate type ion sensor is compact and can easily measure the number of ions.

Although in the above configuration, the mode change switch 41 is provided at the connector 25 and is not visible from the outside, the mode change switch 41 may be provided outside the housing 1aA such that the mode change switch 41 is visible from the outside. With this configuration, the user can easily operate the mode change switch 41 and easily change the time lag.

### Embodiment 3

Embodiment 3 relates to an air-conditioning apparatus 60 provided with the disinfection and virus inactivation device 1A according to Embodiment 1 or 2. The following description is made by referring mainly to the differences in components and processes between Embodiment 3 and Embodiment 1 or 2, and components and processes that will not be described regarding Embodiment 2 are the same as those in Embodiment 1 or 2.

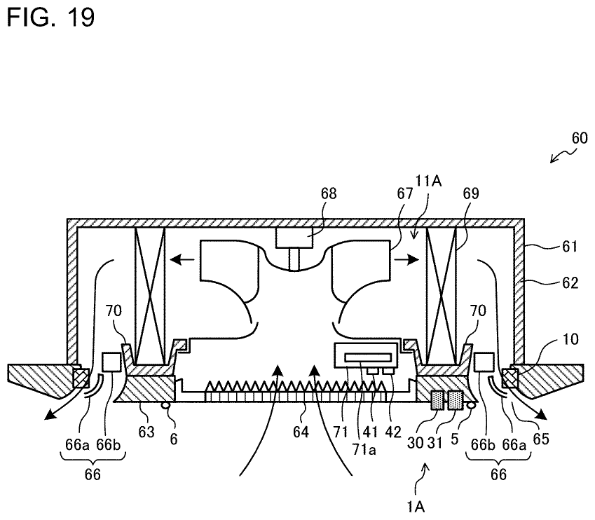

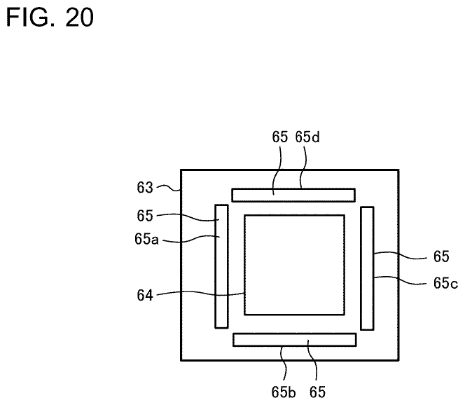

FIG. 19 is a schematic sectional view of an air-conditioning apparatus 60 according to Embodiment 3. FIG. 20 is a schematic view of the air-conditioning apparatus 60 in FIG. 19 as seen from directly below. The air-conditioning apparatus 60 is an indoor unit that is installed in an air-conditioning target space such as an office, and is configured to supply temperature-regulated air to the air-conditioning target space by utilizing a refrigeration cycle that causes refrigerant to circulate. The air-conditioning apparatus 60 performs one or both of a heating operation and a cooling operation as normal operation. The air-conditioning apparatus 60 air-conditions the air-conditioning target space and includes the disinfection and virus inactivation device 1 according to Embodiment 1 or the disinfection and virus inactivation device 1A according to Embodiment 2 to remove bacteria or inactivate viruses in the air-conditioning target space which is determined as the target space S.

The air-conditioning apparatus 60 has a housing 61 embedded in a ceiling, a main body 62 having an opening formed in a lower surface thereof, and a decorative panel 63 that closes the opening of the main body 62. The decorative panel 63 includes a rectangular suction grille 64 in a central part of the decorative panel 63. Four air outlets 65 (65a to 65d) are formed around the suction grille 64 in such a manner as to extend along four sides of the suction grille 64. Each of the air outlets 65 is provided with an airflow direction plate 66 configured to control a direction in which the air flow is blown out through an associated one of the air outlets 65. The air-conditioning apparatus 60 includes, as airflow direction plates 66, an up/down airflow direction plate 66a configured to control the airflow in an up/down direction and a lateral airflow direction plate 66b configured to control the airflow in a lateral direction. Furthermore, in the housing 61, as driving devices each configured to drive an associated one of the up/down airflow direction plate 66a and the lateral airflow direction plate 66b, respective motors (not illustrated) are provided.

A centrifugal air-sending device 67, a motor 68 that drives the centrifugal air-sending device 67, and a heat exchanger 69 that causes heat exchange to be performed between refrigerant flowing therein and air are provided in the housing 61. The centrifugal air-sending device 67 is provided in a central part of the inside of the housing 61 and connected to a shaft extending downward from the motor 68 fixed to a top plate of the housing 61. The heat exchanger 69 is provided around the centrifugal air-sending device 67. Furthermore, a drain pan 70 that receives dew condensation water generated in the heat exchanger 69 is provided in the housing 61 and below the heat exchanger 69. In addition, an electrical box 71 is provided in the housing 61. The electrical box 71 houses a control substrate 71a that controls the operation of the air-conditioning apparatus 60. Although FIG. 19 illustrates an example in which the air-conditioning apparatus 60 is a ceiling-suspended indoor unit, the air-conditioning apparatus 60 is not limited to the ceiling-suspended indoor unit but may be a wall-hung indoor unit.

The air-conditioning apparatus 60 includes the disinfection and virus inactivation device 1A according to Embodiment 2. Specifically, the treatment-mode reception unit 6, the sensing unit 30, and the locus detection unit 31 are provided at the decorative panel 63, and the substance generation unit 10 is provided close to an air outlet 65 of the decorative panel 63. The location of the sensing unit 30 is not limited to a location close to the air outlet 65 of the decorative panel 63, but the sensing unit 30 may be provided at another part of the housing 61. Furthermore, as in Embodiments 1 and 2, the sensing unit 30 may be provided close to the door D.

The heat source unit 13a of the activity reducing unit 13 is the heat exchanger 69. The supply unit 11A includes the centrifugal air-sending device 67, the motor 68, the airflow direction plates 66, and motors (not illustrated) that drive the airflow direction plates 66. The centrifugal air-sending device 67 doubles as the air-sending device 11a of the supply unit 11A. The airflow direction plates 66 have the function of the grille 4a of the supply unit 11A. The indication unit 5 and the treatment-mode reception unit 6 are provided on an outer surface of the decorative panel 63.

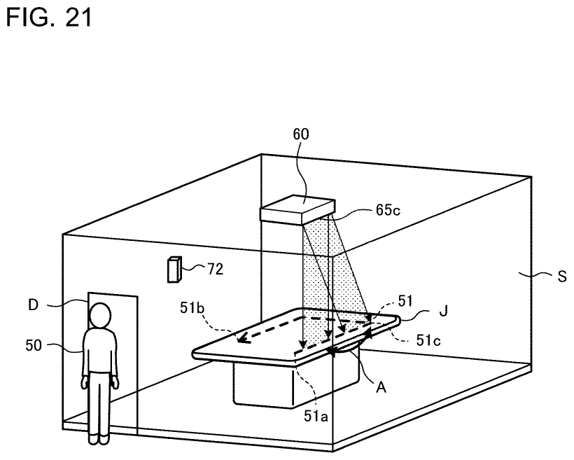

The mode change switch 41 and the communication unit 42 are provided on an outer surface of the electrical box 71. The control substrate 71a in the electrical box 71 has the functions of the controller 20. As illustrated in FIG. 21, the air-conditioning apparatus 60 includes a remote control 72 that enables the user to switch the operation between the heating operation and the cooling operation of the air-conditioning apparatus 60, temperature setting, air volume control, or other operations. The remote control 72 doubles as the treatment-mode inputting unit 7.

FIG. 21 is an explanatory view for a disinfection and virus inactivation operation by the air-conditioning apparatus 60 as illustrated in FIG. 19. The air-conditioning apparatus 60 is installed at a location where it is allowed to supply an air flow toward the equipment J. Actually, in many cases, the air-conditioning apparatus 60 is installed in a room earlier than the equipment J, and the equipment J is thus installed at a location where the air flow from the air-conditioning apparatus 60 reaches the equipment J. Alternatively, at the time of installing the air-conditioning apparatus 60, the location of the air-conditioning apparatus 60 may be determined and the air-conditioning apparatus 60 may be installed based on the location of the equipment J in the target space S. In any case, the air-conditioning apparatus 60 is installed at a certain position such that the movement locus of locations where a person comes into contact with different objects or different portions of an object is located within an air-sending range of the air flow from the air-conditioning apparatus 60.

In the air-conditioning apparatus 60, when the centrifugal air-sending device 67 is rotated by the motor 68, air is sucked into the housing 61 through the suction grille 64, passes through the centrifugal air-sending device 67 and the heat exchanger 69, which forms the heat source unit 13a of the activity reducing unit 13, and is blown out through the air outlets 65. The air flow blown out through the air outlets 65 is an air flow that is temperature-regulated by the heat exchanger 69 and that contains the specific substance generated by the substance generation unit 10. Such an air flow is blown out through the air outlets 65, and the direction in which the air flow is flown out is controlled by the airflow direction plates 66. It should be noted that the controller 20 controls the amount of heat exchange in the heat exchanger 69 according to the treatment mode selected by the treatment-mode selecting unit 22. Thus, the temperature of the air flow blown out through the air outlets 65 is adjusted and set to a temperature suitable for the treatment mode selected by the treatment-mode selecting unit 22.

FIG. 21 illustrates a state in which the air flow is supplied through the air outlet 65c to the movement locus 51. More specifically, FIG. 21 illustrates a state in which the air flow is supplied through the air outlet 65c in such a manner as to trace the movement locus 51 from the starting point 51a to a pass point 51c in a direction indicated by an arrow A. After the supply of the air flow through the air outlet 65c ends, the air flow is supplied toward remaining portions of the movement locus 51 through the air outlet 65b and the air outlet 65a in sequence. Since the air-conditioning apparatus 60 controls, with the airflow direction plates 66, the direction in which the air flow is flown out, the air flow whose straightness and directivity are enhanced and whose temperature is adjusted and set to the temperature at which the activity of the microorganisms decreases can be supplied toward the movement locus 51 of the person in the target space S.

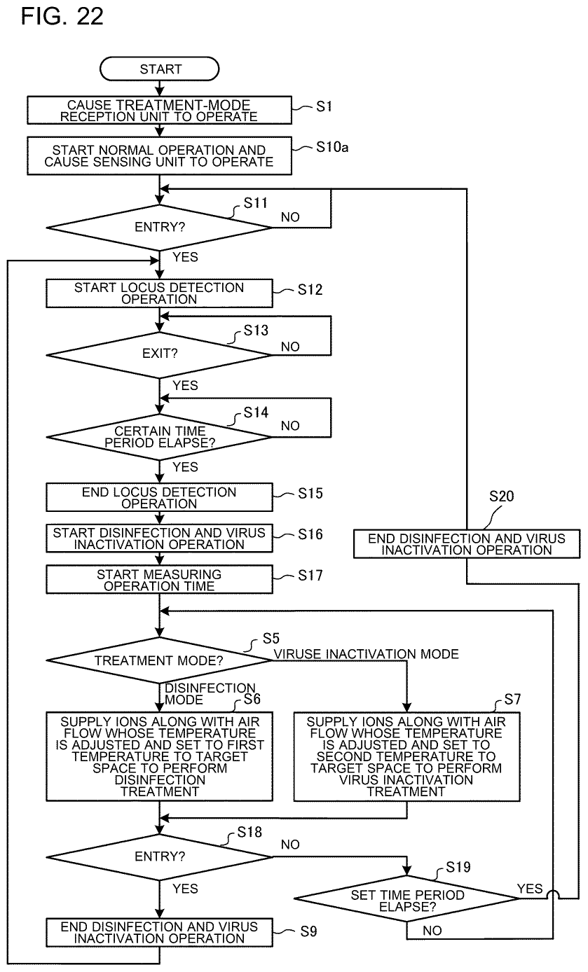

FIG. 22 is an operation flowchart of the air-conditioning apparatus 60 according to Embodiment 3. It will be described with reference to the flowchart of FIG. 22 how the air-conditioning apparatus 60 operates. The following description is made by referring mainly to the differences between the flowchart of FIG. 22 and that of FIG. 18 relating to Embodiment 1.

When a remote switch (not illustrated) set in the target space S is operated to turn on the power supply of the disinfection and virus inactivation device 1A, the controller 20 is activated to cause the treatment-mode reception unit 6 to operate (step S1). Furthermore, the disinfection and virus inactivation device 1A starts the normal operation and in addition causes the sensing unit 30 to operate (step S10a). The normal operation is an operation that is set by the remote control 72, such as the heating operation or the cooling operation. The steps from the above steps onward are the same as in FIG. 18. That is, the flowchart of FIG. 22 is the same as that of FIG. 18 except that in the flowchart of FIG. 22, the normal operation is started when the power supply is turned on.

The air-conditioning apparatus 60 according to Embodiment 3 obtains the same advantages as the disinfection and virus inactivation device 1 according to Embodiment 1 and the disinfection and virus inactivation device 1A according to Embodiment 2, and in addition obtains the following advantages. The air-conditioning apparatus 60 is formed by modifying an existing air-conditioning apparatus 60 originally provided in the target space S such as an office and further incorporating components of the disinfection and virus inactivation device 1A into the air-conditioning apparatus 60 as appropriate. Therefore, the air-conditioning apparatus 60 can efficiently perform disinfection or viral inactivation in the target space S without altering the appearance of the target space S. In the case where an existing air-conditioning apparatus is modified such that an assist louver is attached to the existing air-conditioning apparatus to enhance the directivity and straightness of air passage, the disinfection and virus inactivation effect is improved.

Furthermore, in Embodiment 3, the existing air-conditioning apparatus 60 originally provided in the target space S such as an office may be replaced with an air-conditioning apparatus 60 provided with the disinfection and virus inactivation device 1A. In this case also, as in the case of modifying the existing air-conditioning apparatus 60, the air-conditioning apparatus 60 can efficiently perform disinfection or viral inactivation in the target space S without altering the appearance of the target space S.

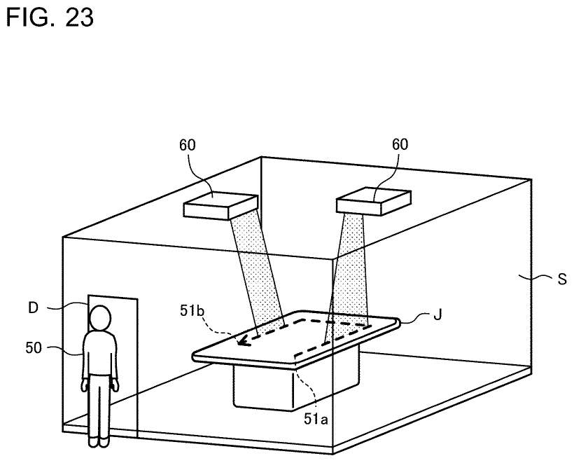

The utilization form of the air-conditioning apparatus 60 according to Embodiment 3 is not limited to a utilization form that a single air-conditioning apparatus 60 is installed in the target space S as illustrated in FIG. 21, and a plurality of air-conditioning apparatuses 60 may be installed in the target space S as illustrated in FIG. 23.

FIG. 23 illustrates a utilization form of the air-conditioning apparatus 60 according to Embodiment 3. Referring to FIG. 23, a plurality of air-conditioning apparatuses 60 are installed in the target space S. In this case, when the plurality of air-conditioning apparatuses 60 perform the same operation, disinfection and viral inactivation can be achieved in a shorter time period than in the case where treatment for the microorganisms in the target space S is carried out with a single air-conditioning apparatus 60. The above “same operation” means that the plurality of air-conditioning apparatuses 60 are the same as each other regarding the mode of operation, such as heating operation or cooling operation, and the treatment mode in the microorganism-activity control unit 21. It should be noted that the plurality of air-conditioning apparatuses 60 may be different regarding the treatment mode in the microorganism-activity control unit 21. That is, one or more of the air-conditioning apparatuses 60 may operate in the disinfection mode, and the other air-conditioning apparatus or apparatuses 60 may operate in the virus inactivating mode. In this case, the air-conditioning apparatuses 60 can perform disinfection and viral inactivation simultaneously.

Furthermore, modifications of Embodiments 1 and 2 can be applied to the air-conditioning apparatus 60 according to Embodiment 3 as appropriate. Specifically, for example, the configuration described above as a modification of Embodiment 2, in which the locus detection unit 31 doubles as the sensing unit 30, can be applied to the disinfection and virus inactivation device 1A according to Embodiment 3.

The present disclosure is not limited to the above embodiments. Needless to say, many modifications and changes can be made to the above embodiments without departing from the scope of the present disclosure. Although an office is cited as an example of the target space S in which the disinfection and virus inactivation device 1A is installed, an ordinary house, a storeroom, and a bathroom can be applied as other examples of the target space S. Furthermore, as other examples of the target space S, for example, a space in a refrigerator and a space in a freezer can be applied.

### REFERENCE SIGNS LIST

1: disinfection and virus inactivation device, 1A: disinfection and virus inactivation device, 1a: housing, 1aA: housing, 2: tubular portion, 2a: lower surface portion, 2a1: exhaust outlet, 3: upper surface portion, 3a: air inlet, 4: grille body, 4a: grille, 4b: air-sending opening, 4c: fin, 4c1: inner end portion, 4c2: outer end portion, 5: indication unit, 6: treatment-mode reception unit, 7: treatment-mode inputting unit, 8: ferrule, 9: air-passage forming component, 9a: air passage, 10: substance generation unit, 11: supply unit, 11A: supply unit, 11a: air-sending device, 12: substance measurement unit, 13: activity reducing unit, 13a: heat source unit, 13a1: heating section, 13a2: cooling section, 13b: temperature sensor, 14: main substrate, 15: bellows portion, 20: controller, 21: microorganism-activity control unit, 22: treatment-mode selecting unit, 25: connector, 25a: hook part, 26: locking part, 30: sensing unit, 30a: transmission unit, 30b: reception unit, 31: locus detection unit, 31a: photographing unit, 31b: image processing unit, 37: air-sending device, 40: driving device, 41: mode change switch, 42: communication unit, 50: person, 51: movement locus, 51a: starting point, 51b: ending point, 51c: pass point, 60: air-conditioning apparatus, 61: housing, 62: main body, 63: decorative panel, 64: suction grille, 65: air outlet, 65a: air outlet, 65b: air outlet, 65c: air outlet, 65d: air outlet, 66: airflow direction plate, 66a: up/down airflow direction plate, 66b: lateral airflow direction plate, 67: centrifugal air-sending device, 68: motor, 69: heat exchanger, 70: drain pan, 71: electrical box, 71a: control substrate, 72: remote control, A: arrow, D: door, J: equipment, O: central part, S: target space

1. A disinfection and virus inactivation device configured to perform a disinfection treatment or an inactivating treatment of microorganisms in a target space, the disinfection and virus inactivation device comprising:
a substance generation unit configured to generate a specific substance to perform the disinfection treatment or the inactivating treatment;
a supply unit configured to generate an air flow and supply the specific substance generated from the substance generation unit into the target space;
an activity reducing unit configured to decrease an activity of the microorganisms;
a treatment-mode selecting unit configured to select, as a treatment mode, a disinfection mode in which disinfection is performed or a virus inactivating mode in which virus inactivation is performed; and
a housing that houses the substance generation unit, the supply unit, the activity reducing unit, and the treatment-mode selecting unit,
wherein the specific substance is supplied to the target space by the supply unit, while the activity of the microorganism is being decreased by the activity reducing unit according to the disinfection mode or the virus inactivating mode that is selected by the treatment-mode selecting unit.

2. The disinfection and virus inactivation device of claim 1, wherein the activity reducing unit includes a heat source unit configured to control a temperature of the microorganisms.

3. The disinfection and virus inactivation device of claim 2, wherein the heat source unit includes a heating section and a cooling section.

4. The disinfection and virus inactivation device of claim 2, further comprising a microorganism-activity control unit configured to control the heat source unit of the activity reducing unit to cause the temperature of the microorganisms to reach a temperature at which the activity of the microorganisms decreases.

5. The disinfection and virus inactivation device of claim 4, wherein
the microorganism-activity control unit has the disinfection mode and the virus inactivating mode as the treatment modes, and is configured to perform a control associated with the mode selected by the treatment-mode selecting unit,
the microorganism-activity control unit is configured to control in the disinfection mode, the heat source unit to cause the temperature of the microorganisms to reach a first temperature set in advance, and to control in the virus inactivating mode, the heat source unit to cause the temperature of the microorganisms to reach a second temperature set in advance.

6. The disinfection and virus inactivation device of claim 5, wherein the first temperature falls within the range of 20 to 25 degrees C., and the second temperature falls within the range of 28 to 30 degrees C.

7. The disinfection and virus inactivation device of claim 2, wherein the heat source unit is provided in a flow passage of the air flow and configured to control the temperature of the microorganisms by controlling a temperature of the air flow.

8. (canceled)

9. The disinfection and virus inactivation device of claim 1, wherein the treatment-mode selecting unit is configured to select the disinfection mode or the virus inactivating mode in response to an input operation by a user.

10. The disinfection and virus inactivation device of claim 1, wherein the treatment-mode selecting unit is configured to select the disinfection mode or the virus inactivating mode based on the number of bacteria or viruses that are detected in the target space.

11. The disinfection and virus inactivation device of claim 1, wherein the treatment-mode selecting unit is configured to select the disinfection mode or the virus inactivating mode based on a season, an indoor environment, a country, or a combination thereof.

12. The disinfection and virus inactivation device of claim 5, wherein the microorganism-activity control unit is configured to alternately perform controls associated with the disinfection mode and the virus inactivating mode.

13. The disinfection and virus inactivation device of claim 1, further comprising a locus detection unit configured to detect a movement locus of locations on portions of equipment provided in the target space where a moving body comes into contact with the portions of the equipment,
wherein the supply unit is configured to supply the specific substance toward the movement locus.

14. An air-conditioning apparatus comprising:
the disinfection and virus inactivation device of claim 1; and
a heat exchanger configured to cause heat exchange to be performed between refrigerant that flows therein and air,
wherein the air-conditioning apparatus is configured to supply the target space with an air flow that is regulated in temperature by the heat exchanger when passing through the heat exchanger and that contains the specific substance.

15. A disinfection and virus inactivation method for performing a disinfection treatment or an inactivating treatment of microorganisms in a target space, the disinfection and virus inactivation method comprising:
generating a specific substance to perform the disinfection treatment or the inactivating treatment;
selecting, as a treatment mode, a disinfection mode in which disinfection is performed or a virus inactivating mode in which virus inactivation is performed; and
supplying the specific substance together with an air flow to the target space, while decreasing an activity of the microorganisms according to the selected disinfection mode or virus inactivating mode.

16. The disinfection and virus inactivation device of claim 1, wherein
the supply unit is an air-sending device, and
the substance generation unit is provided upstream of the supply unit.
